SEQUENCE LISTING (65 sequences)

SEQ ID NO: 1 - DNA, 20 bp, synthetic construct
  source 1..20: mol_type = other DNA
ggcatgctgaaaatccacac

SEQ ID NO: 2 - DNA, 20 bp, synthetic construct
  source 1..20: mol_type = other DNA
cgcaacataggcaatagcac

SEQ ID NO: 3 - DNA, 22 bp, synthetic construct
  source 1..22: mol_type = other DNA
accactactggaatgcacagtt

SEQ ID NO: 4 - DNA, 20 bp, synthetic construct
  source 1..20: mol_type = other DNA
caacgtgcagtgcagagtgc

SEQ ID NO: 5 - DNA, 24 bp, synthetic construct
  source 1..24: mol_type = other DNA
cgaggacaaggaacttgatcccag

SEQ ID NO: 6 - DNA, 23 bp, synthetic construct
  source 1..23: mol_type = other DNA
cctgcctcaggctcccaaagtgc

SEQ ID NO: 7 - RNA, 21 nt, synthetic construct
  source 1..21: mol_type = other RNA
ggagcgagatccctccaaaat

SEQ ID NO: 8 - RNA, 23 nt, synthetic construct
  source 1..23: mol_type = other RNA
ggctgttgtcatacttctcatgg

SEQ ID NO: 9 - RNA, 19 nt, synthetic construct
  source 1..19: mol_type = other RNA
aaggagctcagcatggtca

SEQ ID NO: 10 - RNA, 20 nt, synthetic construct
  source 1..20: mol_type = other RNA
gggcgaggtctgagttgagg

SEQ ID NO: 11 - RNA, 22 nt, synthetic construct
  source 1..22: mol_type = other RNA
cctgacctacccgtgtctctgg

SEQ ID NO: 12 - RNA, 20 nt, synthetic construct
  source 1..20: mol_type = other RNA
accagccacacgcaccattt

SEQ ID NO: 13 - RNA, 22 nt, synthetic construct
  source 1..22: mol_type = other RNA
ctgcttcaaacgatccactcac

SEQ ID NO: 14 - RNA, 18 nt, synthetic construct
  source 1..18: mol_type = other RNA
tgaatccatagacacgcc

SEQ ID NO: 15 - RNA, 30 nt, synthetic construct
  source 1..30: mol_type = other RNA
tcaaatgcagttaagacaaatgcagacgct

SEQ ID NO: 16 - RNA, 25 nt, synthetic construct
  source 1..25: mol_type = other RNA
caccgggcatgctgaaaatccacac

SEQ ID NO: 17 - RNA, 25 nt, synthetic construct
  source 1..25: mol_type = other RNA
aaacgtgtggattttcagcatgccc

SEQ ID NO: 18 - RNA, 25 nt, synthetic construct
  source 1..25: mol_type = other RNA
caccgcagtaaggacgagttccccg

SEQ ID NO: 19 - RNA, 25 nt, synthetic construct
  source 1..25: mol_type = other RNA
aaaccggggaactcgtccttactgc

SEQ ID NO: 20 - RNA, 25 nt, synthetic construct
  source 1..25: mol_type = other RNA
caccggggacttgacgtgttcaagg

SEQ ID NO: 21 - RNA, 25 nt, synthetic construct
  source 1..25: mol_type = other RNA
aaacccttgaacacgtcaagtcccc

SEQ ID NO: 22 - RNA, 25 nt, synthetic construct
  source 1..25: mol_type = other RNA
caccgtgatagtggttggtacatgt

SEQ ID NO: 23 - RNA, 25 nt, synthetic construct
  source 1..25: mol_type = other RNA
aaacacatgtaccaaccactatcac

SEQ ID NO: 24 - RNA, 25 nt, synthetic construct
  source 1..25: mol_type = other RNA
caccgcacgcacagcacgtccaccg

SEQ ID NO: 25 - RNA, 25 nt, synthetic construct
  source 1..25: mol_type = other RNA
aaaccggtggacgtgctgtgcgtgc

SEQ ID NO: 26 - RNA, 25 nt, synthetic construct
  source 1..25: mol_type = other RNA
caccggaacgggagcgccgcccgaa

SEQ ID NO: 27 - RNA, 25 nt, synthetic construct
  source 1..25: mol_type = other RNA
aaacttcgggcggcgctcccgttcc

SEQ ID NO: 28 - RNA, 25 nt, synthetic construct
  source 1..25: mol_type = other RNA
caccgtcggtcacactcaggccgtg

SEQ ID NO: 29 - RNA, 25 nt, synthetic construct
  source 1..25: mol_type = other RNA
aaaccacggcctgagtgtgaccgac

SEQ ID NO: 30 - RNA, 25 nt, synthetic construct
  source 1..25: mol_type = other RNA
caccgcgcaacataggcaatagcac

SEQ ID NO: 31 - RNA, 25 nt, synthetic construct
  source 1..25: mol_type = other RNA
aaacgtgctattgcctatgttgcgc

SEQ ID NO: 32 - RNA, 25 nt, synthetic construct
  source 1..25: mol_type = other RNA
caccgcatgtgccttaaggggatgg

SEQ ID NO: 33 - RNA, 25 nt, synthetic construct
  source 1..25: mol_type = other RNA
aaacccatccccttaaggcacatgc

SEQ ID NO: 34 - RNA, 25 nt, synthetic construct
  source 1..25: mol_type = other RNA
caccgggacacgttcacttacgcca

SEQ ID NO: 35 - RNA, 25 nt, synthetic construct
  source 1..25: mol_type = other RNA
aaactggcgtaagtgaacgtgtccc

SEQ ID NO: 36 - RNA, 25 nt, synthetic construct
  source 1..25: mol_type = other RNA
caccgtggatgaggtccatgcagtg

SEQ ID NO: 37 - RNA, 25 nt, synthetic construct
  source 1..25: mol_type = other RNA
aaaccactgcatggacctcatccac

SEQ ID NO: 38 - RNA, 25 nt, synthetic construct
  source 1..25: mol_type = other RNA
caccgtcacggactatagttaccag

SEQ ID NO: 39 - RNA, 25 nt, synthetic construct
  source 1..25: mol_type = other RNA
aaacctggtaactatagtccgtgac

SEQ ID NO: 40 - DNA, 21 bp, synthetic construct
  source 1..21: mol_type = other DNA
tggccagtgtggattttcagc

SEQ ID NO: 41 - RNA, 19 nt, synthetic construct
  source 1..19: mol_type = other RNA
tggccagtggattttcagc

SEQ ID NO: 42 - DNA, 21 bp, synthetic construct
  source 1..21: mol_type = other DNA
cctgtgctattgcctatgttg

SEQ ID NO: 43 - DNA, 20 bp, synthetic construct
  source 1..20: mol_type = other DNA
cctgtgtattgcctatgttg

SEQ ID NO: 44 - DNA, 30 bp, synthetic construct
  source 1..30: mol_type = other DNA
cttccagcgttttcaaacatgactcgaact

SEQ ID NO: 45 - DNA, 32 bp, synthetic construct
  source 1..32: mol_type = other DNA
acatttcaccaaatgccgaagcattcaacaag

SEQ ID NO: 46 - DNA, 33 bp, synthetic construct
  source 1..33: mol_type = other DNA
aaatcgaatgcagttaagacaaatgcagacgct

SEQ ID NO: 47 - DNA, 16 bp, synthetic construct
  source 1..16: mol_type = other DNA
cctattgcctatgttg

SEQ ID NO: 48 - DNA, 56 bp, synthetic construct
  source 1..56: mol_type = other DNA
acctgttcctggccttgagtgagcacaatccagctggaaatatcttcatctctccc

SEQ ID NO: 49 - DNA, 46 bp, synthetic construct
  source 1..46: mol_type = other DNA
acctgttcctggccttgagtgagctggaaatatcttcatctctccc

SEQ ID NO: 50 - DNA, 51 bp, synthetic construct
  source 1..51: mol_type = other DNA
ccctggctcatccaccgctaccgatatctgggtcatcgcttccaaggtacg

SEQ ID NO: 51 - DNA, 52 bp, synthetic construct
  source 1..52: mol_type = other DNA
ccctggctcatccaccgctaccgattatctgggtcatcgcttccaaggtacg

SEQ ID NO: 52 - DNA, 50 bp, synthetic construct
  source 1..50: mol_type = other DNA
agcagctccatgtcggtgtcagagctgaaggcaaagatcgcccagaagat

SEQ ID NO: 53 - DNA, 52 bp, synthetic construct
  source 1..52: mol_type = other DNA
agcagctccatgtcggtgtcagagcattgaaggcaaagatcgcccagaagat

SEQ ID NO: 54 - DNA, 53 bp, synthetic construct
  source 1..53: mol_type = other DNA
agcagctccatgtcggtgtcagagctgaaggcaaagatcgcccagaagatcgg

SEQ ID NO: 55 - DNA, 54 bp, synthetic construct
  source 1..54: mol_type = other DNA
agcagctccatgtcggtgtcagagcctgaaggcaaagatcgcccagaagatcgg

SEQ ID NO: 56 - DNA, 51 bp, synthetic construct
  source 1..51: mol_type = other DNA
tatttgaatctacagtgccctcggcattctacgataagtgtccaatcggca

SEQ ID NO: 57 - DNA, 47 bp, synthetic construct
  source 1..47: mol_type = other DNA
tatttgaatctacagtgccctcggctacgataagtgtccaatcggca

SEQ ID NO: 58 - DNA, 50 bp, synthetic construct
  source 1..50: mol_type = other DNA
tatttgaatctacagtgccctcgcattctacgataagtgtccaatcggca

SEQ ID NO: 59 - DNA, 40 bp, synthetic construct
  source 1..40: mol_type = other DNA
tatttgaatctacagtgctacgataagtgtccaatcggca

SEQ ID NO: 60 - DNA, 32 bp, synthetic construct
  source 1..32: mol_type = other DNA
tatttgaatctacgataagtgtccaatcggca

SEQ ID NO: 61 - DNA, 53 bp, synthetic construct
  source 1..53: mol_type = other DNA
ctatgttgcgttagtgactctactgatctgggttcctgtgaaagttctcctgc

SEQ ID NO: 62 - DNA, 48 bp, synthetic construct
  source 1..48: mol_type = other DNA
ctatgttgcgttagtgactctactgggttcctgtgaaagttctcctgc

SEQ ID NO: 63 - DNA, 52 bp, synthetic construct
  source 1..52: mol_type = other DNA
ctatgttgcgttagtgactctactatctgggttcctgtgaaagttctcctgc

SEQ ID NO: 64 - DNA, 54 bp, synthetic construct
  source 1..54: mol_type = other DNA
ctatgttgcgttagtgactctaccccatctgggttcctgtgaaagttctcctgc

SEQ ID NO: 65 - DNA, 55 bp, synthetic construct
  source 1..55: mol_type = other DNA
ctatgttgcgttagtgactctactgtgatctgggttcctgtgaaagttctcctgc

(19) United States
(12) Patent Application Publication
(10) Pub. No.: US 2026/0103682 A1
(43) Pub. Date: Apr. 16, 2026

(54) GENETICALLY MODIFIED VERO CELLS

(71) Applicant: Washington University, St. Louis, MO (US)

(72) Inventors: Siyuan Ding, St. Louis, MO (US); Qiru Zeng, St. Louis, MO (US); Yinxing Zhu, St. Louis, MO (US)

(73) Assignee: Washington University, St. Louis, MO (US)

(21) Appl. No.: 19/356,035

(22) Filed: Oct. 10, 2025

Related U.S. Application Data
(60) Provisional application No. 63/705,514, filed on Oct. 10, 2024.

(51) Int. Cl. C12N 5/071; C12N 9/22; C12N 15/11; C12N 15/86

(52) U.S. Cl. CPC C12N 5/0686; C12N 9/226; C12N 15/11; C12N 15/86; C12N 2310/20; C12N 2510/00; C12N 2720/12343

(57) ABSTRACT

Among the various aspects of the present disclosure is the provision of genetically modified Vero cells. The present teachings include compositions for a rotavirus reverse genetics system that can include SERPINB1 knockout cells combined with a helper plasmid. The present teachings also include a vaccine-producing cell substrate that can include Vero cells with disrupted TMEM236 and method of use thereof.

### CROSS-REFERENCE TO RELATED APPLICATIONS

This application claims the benefit of priority to U.S. Provisional Application Ser. No. 63/705,514 filed on Oct. 10, 2024, the content of which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer readable form comprising nucleotide and/or amino acid sequences of the present invention (file name “021138-US-NP_Sequence-listing” created on 9 Oct. 2025; 58,930 bytes). The subject matter of the Sequence Listing is incorporated herein by reference in its entirety.

### FIELD OF THE INVENTION

The present disclosure generally relates to genetically modified cells related to vaccine efficacy.

### BACKGROUND OF THE INVENTION

Rotaviruses (RVs) belong to the Sedoreoviridae family, a group of double-stranded RNA (dsRNA) non-enveloped viruses. The genome of RV consists of 11 segments (genes 1-11), which encode 6 structural proteins (VP1-4, VP6, and VP7) and 6 nonstructural proteins (NSP1-6). Although there are two vaccines available globally, i.e., Rotarix® (RV1, GSK), and RotaTeq® (RV5, Merck), RV infection results in the death of more than 128,500 children per year worldwide, highlighting the need for more efficacious vaccines.

The first reverse genetics system for RV was described in 2006, in which a human RV strain was used as a helper virus. The transformative plasmid-only-based reverse genetics system for RV was developed in 2017. In this system, each of 11 RV cDNAs is driven by a T7 promoter and ends with a hepatitis delta virus ribozyme sequence. The 11 rescue plasmids are transfected into BHK-2 T7 cells along with plasmids encoding two subunits of the vaccinia virus capping enzyme (D1R and D12L) and a Nelson Bay reovirus fusion-associated small transmembrane protein (FAST). Because RV does not replicate efficiently in BHK-T7 cells, MA104 cells, which are highly susceptible to RV infection, are used as an overlay to propagate the rescued virus.

The limited efficiency in recovering these hard-to-rescue strains reveals a critical gap for RV reverse genetics systems. Multiple potential means to increase rescue efficiency have been recently discussed, one of which is an optimized MA104 cell substrate for the overlaying step.

Whole genome loss-of-function CRISPR/Cas9 screens have been used to identify pro-viral host factors for RV, influenza, SARS-CoV-2, hepatitis B virus, hepatitis C virus, flavivirus, and murine norovirus, but fewer studies have focused on the anti-viral host factors.

### SUMMARY OF THE INVENTION

Among the various aspects of the present disclosure is the provision of genetically modified Vero cells.

Briefly, therefore, the present disclosure is directed to genetically modified Vero cells that enhance vaccine efficacy.

The present teachings include compositions for a rotavirus reverse genetics system. The system comprises SERPINB1 knockout cells or TMEM236 knockout cells. In some aspects, the system can also include a helper plasmid comprising a C3P3-G3 plasmid, a layer of BHK-T7 cells, or any combination thereof. In some aspects, the SERPINB1 knockout cell or the TMEM236 knockout cell comprises a Vero cell. In some aspects, the SERPINB1 or TMEM236 knockout cell is generated using a CRISPR/Cas9 system comprising a single-guide RNA (sgRNA) selected from SEQ ID NO: 1 and SEQ ID NO: 2. In some aspects, the SERPINB1 knockout cell or the TMEM236 knockout cell are infected with a live-attenuated vaccine. In some aspects, the live-attenuated vaccine comprises a Rotavirus vaccine or an Ebola vaccine.

The present teachings also include a vaccine-producing cell substrate. The substrate comprises a Vero cell with disrupted SERPINB1 or TMEM236. In one aspect, the Vero cells are transfected with a CRISPR/Cas9 plasmid comprising a sgRNA selected from SEQ ID NO: 1 or SEQ ID NO: 2, to disrupt SERPINB1 or TMEM236. In another aspect, the vaccine-producing cell substrate increases production of a live-attenuated vaccine. In another aspect, the live-attenuated vaccine comprises a rotavirus (RV) vaccine or an Ebola vaccine.

The present teachings also include a method of increasing production of a vaccine in a cell. The method comprises disrupting an anti-viral protein in the vaccine producing cell; introducing a helper component to the vaccine producing cell; infecting the vaccine producing cells with a live-attenuated vaccine; and collecting the vaccine from the vaccine producing cell. In some aspects, the vaccine producing cell is a Vero cell. In some aspects, the anti-viral protein disrupted in the vaccine producing cell is TMEM236 or SERPINB1. In some aspects, TMEM236 or SERPINB1 is knocked out of the vaccine producing cell using a CRISPR/Cas9 system comprising a sgRNA selected from SEQ ID NO: 1 and SEQ ID NO: 2. In some aspects, the helper component comprises a C3P3-G3 plasmid, a BHK-T7 cell, or any combination thereof. In some aspects, the live-attenuated vaccine comprises a Rotavirus vaccine or an Ebola vaccine. In some aspects, vaccine production increase by 2-fold to 10-fold.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a schematic flowchart for RB live/dead-based loss-of-function CRISPR screening approach.

FIG. 1B is a graph showing the scores of −Log₁₀ (p value) of the top hits in the CRISPR screen (FIG. 1A), sorted on the x-axis by gene Rank.

FIG. 1C is a graph showing the scores of Log₂ (fold change) of the top hits in the CRISPR screen (FIG. 1A), sorted on the x-axis by gene Rank.

FIG. 2A is a schematic flowchart for RV FACS-based loss-of-function CRISPR screening approach.

FIG. 2B is a graph showing the scores of −Log 10 (p value) of the top hits in the CRISPR screen (FIG. 2A), sorted on the x-axis by gene Rank.

FIG. 2C is a graph showing the scores of Log 2 (fold change) of the top hits in the CRISPR screen (FIG. 2A), sorted on the x-axis by gene Rank.

FIG. 2D is a graph showing wild-type (WT) and indicated genes knockout MA104 cells were infected with rRRV-GFP (MOI=1), and GFP signal was measured by flow cytometry analysis. Experiments were repeated at least three times in triplicates. ****p<0.0001 using one-way ANOVA with Tukey's post hoc test.

FIG. 2E is a chart showing protein function analysis by Panther Classification System.

FIG. 3A is a schematic showing the regions of sgRNA-targeted SERPINB1 genes amplified and examined by Sanger sequencing.

FIG. 3B is a schematic showing the regions of sgRNA-targeted TMEM236 genes amplified and examined by Sanger sequencing.

FIG. 3C is a graph showing increased rotavirus (RRV) replication in WT, SERPINB1 KO, and TMEM236 KO MA104 cells infected with RRV (MOI=0.01) in the presence of trypsin (0.5 μg/ml) for 48 h. Viral titers were determined by plaque forming unit (PFU) assays. Data represents the average of three experiments; error bars indicate SEM (one-way ANOVA with Tukey's post hoc test; ****P<0.0001).

FIG. 3D is a graph showing the diameter of plaque size of WT, SERPINB1 KO, and TMEM236 KO MA104 cells infected with RRV (MOI=0.01) in the presence of trypsin (0.5 ug/ml) for 4 days. The diameter of the plaque size was determined by calculating the diameter of the plaque. Data represents the average of three experiments. Error bars indicate SEM; one-way ANOVA with Tukey's post hoc test; ****p<0.0001).

FIG. 3E is a graph showing WT, SERPINB1 KO, and TMEM236 KO MA104 cells infected with RRV (MOI=0.01) for indicated timepoints. Viral titers were measured by focus forming unit (FFU) assays. Data represents the average of three experiments; error bars indicate SEM; two-way ANOVA with Tukey's post hoc test; ****p<0.0001). Without indication, p>0.05, not significant.

FIG. 3F is a graph showing the diameter of plaque size of WT, SERPINB1 KO, and TMEM236 KO MA104 cells infected with UK (MOI=0.01) in the presence of trypsin (0.5 ug/ml) for 4 days. The diameter of the plaque size was determined by calculating the diameter of the plaque. Data represents the average of three experiments. Error bars indicate SEM; one-way ANOVA with Tukey's post hoc test; ****p<0.0001).

FIG. 3G is a graph showing the diameter of plaque size of WT, SERPINB1 KO, and TMEM236 KO MA104 cells infected with WI61 (MOI=0.01) in the presence of trypsin (0.5 ug/ml) for 4 days. The diameter of the plaque size was determined by calculating the diameter of the plaque. Data represents the average of three experiments. Error bars indicate SEM; one-way ANOVA with Tukey's post hoc test; ****p<0.0001).

FIG. 3H is a graph showing increased virus replication in WT, SERPINB1 KO, and TMEM236 KO MA104 cells infected with UK (MOI=0.01) in the presence of trypsin (0.5 μg/ml) for 48 h. Viral titers were determined by focus forming unit (FFU) assays. Data represents the average of three experiments; error bars indicate SEM (one-way ANOVA with Tukey's post hoc test; *P<0.05; ****P<0.0001).

FIG. 3I is a graph showing increased virus replication in WT, SERPINB1 KO, and TMEM236 KO MA104 cells infected with WI61 (MOI=0.01) in the presence of trypsin (0.5 μg/ml) for 48 h. Viral titers were determined by focus forming unit (FFU) assays. Data represent the average of three experiments; error bars indicate SEM; one-way ANOVA with Tukey's post hoc test; *P<0.05; **P<0.01.

FIG. 4A is a graph showing viral titers from an FFU assay. A SA11 viruses were rescued on BHK-T7 cells and then overlayed with WT, NV, SERPINB1, and TMEM236 knockout MA104 cells. Data represent the average of three experiments; error bars indicate SEM; one-way ANOVA with Tukey's post hoc test; ***P<0.001, ****P<0.0001). Without indication, P>0.05, not significant.

FIG. 4B is a set of flow cytometry plots showing BHK-T7 cells were transfected with pT7-SA11-NSP3-GFP plasmid and C3P1-G1 or C3P3-G3 helper plasmids.

FIG. 4C is a graph showing the percent of GFP+ BHK-T7 cells which were transfected with pT7-SA11-NSP3-GFP plasmid and C3P1-G1 or C3P3-G3 helper plasmids. Data represent the average of three experiments; error bars indicate SEM; one-way ANOVA with Tukey's post hoc test; ****P<0.0001. Without indication, P>0.05, not significant.

FIG. 4D is a graph showing the mean fluorescence intensity of BHK-T7 cells which were transfected with pT7-SA11-NSP3-GFP plasmid and C3P1-G1 or C3P3-G3 helper plasmids. Data represent the average of three experiments; error bars indicate SEM; one-way ANOVA with Tukey's post hoc test; **P<0.01. Without indication, P>0.05, not significant.

FIG. 4E is a graph showing viral titer determined by FFU assay. SA11 viruses were rescued by co-transfected with C3P3-G1 or C3P3-G3 helper plasmids. Error bars indicate SEM; student's t test; **p<0.01.

FIG. 5A is a schematic diagram of a genetically engineered pT7-D6/2-NSP3 plasmid that encodes NLuc with nucleotide positions indicated in gene segment 7. For generation of an NSP6 deletion virus, four start codons of NSP6 were mutated in gene segment 11. UTR, untranslated region; P2A, self-cleaving P2A peptide gene of porcine teschovirus-1.

FIG. 5B is an image showing viral dsRNA extracted from sucrose cushion-concentrated virus, separated on a 10% polyacrylamide gel, and then stained with ethidium bromide. The dsRNA segment numbers are indicated and the position of the engineered gene segment 7 is marked with an asterisk.

FIG. 5C is a graph showing luciferase activity of rD6/2-2 g, rD6/2-2g-NLuc, and rD6/2-2g-NSP6-Del-NLuc. MA104 cells were infected with 10-fold serially diluted rD6/2-2 g or rD6/2-2g-NLuc or rD6/2-2g-NSP6-Del-NLuc. Cells were harvested at 48 hpi and the luciferase activity was determined by Nano-Glo® luciferase assay. Results are expressed as the mean luminescence of triplicates and error bars show SEM; two-way ANOVA with Turkey's post hoc test, ****p<0.0001).

FIG. 5D is a graph showing NSP5 mRNA in MA104 cells infected with rD6/2-2g-NLuc and rD6/2-2g-NSP6-Del-NLuc (MOI=0.01) in the presence of trypsin (0.5 μg/ml) and harvested at the indicated time points. The viral mRNA level was determined by RT-qPCR assay and normalized to that of GAPDH. Data are the average of three experiments, error bars indicate SEM (two-way ANOVA with Turkey's post hoc test; without indication, P>0.05, not significant.

FIG. 5E is a graph showing viral titer of MA104 cells infected with rD6/2-2g-NLuc and rD6/2-2g-NSP6-Del-NLuc (MOI=0.01) in the presence of trypsin (0.5 μg/ml) and harvested at the indicated time points. Virus titer was determined by FFU assay. Data are the average of three experiments, error bars indicate SEM; two-way ANOVA with Turkey's post hoc test; *P<0.05, **P<0.01.

FIG. 5F is a graph showing the diarrhea rate of five-day-old C57BL/6 mice (n=6, 8) which were orally inoculated with 3.5×10³ FFUs of rD6/2-2g-NLuc or rD6/2-2g-NSP6-Del-NLuc. The diarrhea rate was monitored from 1 to 8 dpi.

FIG. 5G is a set of representative images of rD6/2-2g-NLuc (left) and rD6/2-2g-NSP6-Del-NLuc (right) five-day-old C57BL/6 mice showing luciferase signals of NLucencoding RV-infected animals at 8 dpi.

FIG. 5H is a graph quantifying the bioluminescent signal of rD6/2-2g-NLuc (n=6) and rD6/2-2g-NSP6-Del-NLuc (n=8) of five-day-old C57BL/6 mice. Bioluminescent signal is expressed in photons per second per square centimeter per steradian (p/sec/cm2/sr). Error bars indicate SEM; two-way ANOVA with Turkey's post hoc test; *P<0.05; without indication, P>0.05, not significant.

FIG. 6A is a graph of WT and knockout MA104 cells in T25 flask were infected with Rotarix (MOI=0.01) in the presence of trypsin (0.5 μg/ml) for 96 h. Viral titers were determined by FFU assays. Data represents the average of three experiments; error bars indicate SEM; one-way ANOVA with Tukey's post hoc test; ****P<0.0001.

FIG. 6B is a graph of WT and knockout MA104 cells in T25 flask were infected with RotaTeq (MOI=0.01) in the presence of trypsin (0.5 μg/ml) for 96 h. Viral titers were determined by FFU assays. Data represents the average of three experiments; error bars indicate SEM; one-way ANOVA with Tukey's post hoc test; ****P<0.0001.

FIG. 6C is a graph showing WT and TMEM236 knockout Vero cells in T25 flask were infected with Rotarix (MOI=0.01) in the presence of trypsin (0.5 μg/ml) for 96 h (rVSVZEBOV for 48 h). Viral titers were determined by FFU assays. Data represent the average of three experiments; error bars indicate SEM; one-way ANOVA with Tukey's post hoc test; ****P<0.0001).

FIG. 6D is a graph showing WT and TMEM236 knockout Vero cells in T25 flask were infected with RotaTeq (MOI=0.01) in the presence of trypsin (0.5 μg/ml) for 96 h (rVSVZEBOV for 48 h). Viral titers were determined by FFU assays. Data represent the average of three experiments; error bars indicate SEM; one-way ANOVA with Tukey's post hoc test; ****P<0.0001).

FIG. 6E is a graph showing WT and TMEM236 knockout Vero cells in T25 flask were infected with rVSVZEBOV (MOI=0.01) in the presence of trypsin (0.5 μg/ml) for 96 h (rVSVZEBOV for 48 h). Viral titers were determined by FFU assays. Data represent the average of three experiments; error bars indicate SEM; one-way ANOVA with Tukey's post hoc test; ****P<0.0001).

FIG. 6F is a graph showing WT, TMEM236 knockout, and complemented (expressing V5-TMEM236 in TMEM236 knockout cells) Vero cells in T25 flask were infected with RotaTeq (MOI=0.01) in the presence of trypsin (0.5 μg/ml) for 96 h (rVSVZEBOV for 48 h). Viral titers were determined by FFU assays. Data represent the average of three experiments; error bars indicate SEM; one-way ANOVA with Tukey's post hoc test; ****P<0.0001).

FIG. 7A is a set of flow cytometry plots of mock infection (left) and rRRV-GFP virus infected (right) MA104 cells at an MOI of 5 for 8 hours.

FIG. 7B is a set of immunofluorescent images of MA104-Cas9 cells infected with mock (left) and rRRV-GFP (right) at an MOI of 5 for 8 hours.

FIG. 7C is a set of flow cytometry plots showing WT and pooled gene knockout MA104 cells infected with rRRV-GFP virus at an MOI of 1 for 8 hours. Then cells were analyzed by flow cytometry.

FIG. 8A is a set of images of RRV (top) and UK (bottom) infected WT and gene knockout MA104 cells at an MOI of 0.01 for 24 hours. Cells are then stained with crystal violet.

FIG. 8B is a graph showing enrichment of human SERPINB1 in different tissues based on human gene atlas.

FIG. 8C is a graph showing enrichment of human TMEM236 in different tissues based on human gene atlas.

FIG. 9A is a set of graphs showing transcript levels of SERPINB1 (left) and TMEM236 (right) of WT, SERPINB1 KO, and TMEM236 KO MA104 cells were analyzed by RT-qPCR. Data represents the average of three experiments; error bars indicate SEM; t test; ****, p<0.0001).

FIG. 9B is a set of flow cytometry plots of WT, SERPINB1 KO, and TMEM236 KO MA104 cells were stained with CSFE (5 μM) and seeded into 6 well plates. Cells were harvested at the indicated time intervals and analyzed by flow cytometry.

FIG. 9C is a graph showing mean fluorescence intensity (MFI) of CFSE positive cells examined at each time point. Data represents the average of three experiments; error bars indicate SEM; two-way ANOVA with Tukey's post hoc test; ns, not significant.

FIG. 10A is a set of images of WT (left), SERPINB1 KO (middle), and TMEM236 KO (right) MA104 cells infected with RRV (MOI=0.01) in the presence of trypsin (0.5 μg/ml) for 4 days. The cells were stained with neutral red for 3 hours.

FIG. 10B is a graph quantifying NSP5 mRNA from WT (left), SERPINB1 KO (middle), and TMEM236 KO (right) MA104 cells infected with RRV (MOI=0.01) for 24 hours. Total RNA was analyzed by RT-qPCR. Data represent the average of three experiments; error bars indicate SEM; t test; ****, P<0.0001.

FIG. 11 is a graph and a set of corresponding immunofluorescent images of WT and TMEM236 KO MA104 cells transfected with vector control (Vec) or V5-TMEM236 plasmid for 24 hours and then infected with RRV (MOI=0.01) for 24 hours. Viral titer was determined by FFU assay. The expression of V5-TMEM236 was confirmed by immunofluorescence assay with an anti-V5 antibody.

FIG. 12 is a set of images of WT (left), SERPINB1 KO (middle), and TMEM236 KO (right) MA104 cells infected with UK (top) and WI61 (bottom) (MOI=0.01) in the presence of trypsin (0.5 μg/ml) for 4 days. The cells were stained with neutral red for 3 hours.

FIG. 13 is a graph showing plaque size of rVSV-SARS-CoV-2 in WT, SERPINB1 and TMEM236 knockout cells. WT and gene knockout MA104 cells were infected with rVSV-SARS-CoV-2 at an MOI of 0.01 for 3 days. The cells were stained with neutral red for 3 hours and plaque sizes were determined. Plates were scanned on a biomolecular imager and GFP level was quantified at 72 hpi. Data represents the average of three experiments; error bars indicate SEM (one-way ANOVA with Tukey's post hoc test; ****, P<0.0001).

FIG. 14 is a graphical representation of Sanger sequencing of an NSP6-deletion virus. Viral RNA of rD6/2-2g-NSP6-Del-NLuc was extracted and the gene segment 11 was amplified for Sanger sequencing. The yellow highlighted areas represent the possible start codons.

FIG. 15 is a graphical representation showing sequencing of a single clone of TMEM236 knockout cells. Genome of single clone of TMEM236 knockout Vero cells was extracted and TMEM236 gene was amplified and sequenced by Sanger sequencing.

FIG. 16 is a western blot showing V5 (top) and GAPDH (bottom) expression in WT and TMEM236 KO Vero cells.

FIG. 17 is a graph showing RRV infection of WT, SERPINB1 KO, TMEM236 KO, and double knockout MA104 cells at an MOI of 0.01 for the indicated time points. Virus titers were determined by FFU assays. Data represent the average of three experiments; error bars indicate SEM; two-way ANOVA with Tukey's post hoc test; ****, P<0.0001.

FIG. 18A is a schematic showing the experimental design for producing viruses from WT or knockout Vero cells.

FIG. 18B is a graph showing the fold change in virus titer from WT and knockout Vero cells.

FIG. 19A is an image comparing a reference sequence (SEQ ID NO: 48) and altered sequence (SEQ ID NO: 49) for SERPINB1.

FIG. 19B is an image comparing a reference sequence (SEQ ID NO: 50) and altered sequence (SEQ ID NO: 51) for EMX2.

FIG. 19C is an image comparing a reference sequence (SEQ ID NO: 52; SEQ ID NO: 54) and altered sequence (SEQ ID NO: 53; SEQ ID NO: 55) for ISG15.

FIG. 19D is an image comparing a reference sequence (SEQ ID NO: 56) and altered sequence 1 (SEQ ID NO: 57), 2 (SEQ ID NO: 58), 3 (SEQ ID NO: 59), 4 (SEQ ID NO: 60) for EVAC1.

FIG. 19E is an image showing a reference sequence (SEQ ID NO: 61) and altered sequences (SEQ ID NO: 62; SEQ ID NO: 63; SEQ ID NO: 64; SEQ ID NO: 65) for TMEM236.

### DETAILED DESCRIPTION OF THE INVENTION

The present disclosure is based, at least in part, on the discovery of the most significant antiviral genes in Vero cells by a genome-wide CRISPR screen. Because Vero cells are widely used as the cell substrate for the propagation of multiple live-attenuated vaccines including rotavirus, gene-edited Vero cells with Rotarix (GSK), RotaTeq (Merck) vaccine strains are used to demonstrate improved vaccine production efficiency. Other live-attenuated vaccines include Ebola, where gene-edited cells propagate vaccine strains such as rVSV-ZEBOV. As shown herein, CRISPR/Cas9 screens identify key host factors that enhance rotavirus reverse genetics efficacy and vaccine production.

One of the two most widely used oral live-attenuated rotavirus (RV) vaccine is ROTARIX. It is also known as RV1 and is manufactured by GlaxoSmithKline Biologicals (GSK). Rotarix vaccine strain is originally derived from the human 89-12 strain that belongs to G1P type and is propagated on Vero cells. Unfortunately, there has been shortage of Rotarix twice in the past 6 years (2018 and 2021). The overall objective of the present disclosure is to increase the titer of Rotarix by modifying Vero cells. When achieved, this greatly enhances the global yield and coverage of Rotarix and helps reduce RV infection-associated morbidity and mortality.

### Vaccine Modulation Agents

As described herein, G1P expression has been implicated in various diseases, disorders, and conditions. As such, modulation of G1P (e.g., modulation of G1P on Vero cells) can be used for treatment of such conditions. A G1P modulation agent can modulate G1P response or induce or inhibit G1P. G1P modulation can comprise modulating the expression of G1P on cells, modulating the quantity of cells that express G1P, or modulating the quality of the G1P cells.

Vaccine modulation agents can be any composition or method that can modulate G1P expression on cells (e.g., induce G1P expression on Vero cells). For example, a vaccine modulation agent can be an activator, an inhibitor, an agonist, or an antagonist. As another example, the vaccine modulation can be the result of gene editing.

A vaccine modulation agent can be a G1P antibody (e.g., a monoclonal antibody to G1P).

A vaccine modulating agent can be an agent that induces or inhibits progenitor cell differentiation into G1P expressing cells.

A vaccine modulating agent can be an agent that increases vaccine production. For example, a vaccine modulating agent can be a helper plasmid, such as C3P3-G1, C3P3-G2, and C3P3-G3. A vaccine modulating agent can be a plasmid or BHK-T7 cell, which increase T7 DNA-dependent RNA polymerase.

SERPINB1 or TMEM236 Signal Reduction, Elimination, or Inhibition by Small Molecule Inhibitors, shRNA, siRNA, or ASOs

As described herein, a vaccine modulation agent can be used for use in vaccine therapy. A vaccine modulation agent can be used to reduce/eliminate or enhance/increase SERPINB1 or TMEM236 signals. For example, a vaccine modulation agent can be a small molecule inhibitor of SERPINB1 or TMEM236. As another example, a vaccine modulation agent can be a short hairpin RNA (shRNA). As another example, a vaccine modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

### SERPINB1 or TMEM236 Inhibiting Agent

One aspect of the present disclosure provides for targeting of SERPINB1 or TMEM236, their receptor, or their downstream signaling. The present disclosure provides methods of treating or preventing a disease based on the discovery of the most significant antiviral genes in Vero cells.

As described herein, inhibitors of SERPINB1 or TMEM236 (e.g., antibodies, fusion proteins, small molecules) can reduce or prevent a disease. A SERPINB1 or TMEM236 inhibiting agent can be any agent that can inhibit SERPINB1 or TMEM236, downregulate SERPINB1 or TMEM236, or knockdown SERPINB1 or TMEM236.

As an example, a SERPINB1 or TMEM236 inhibiting agent can inhibit SERPINB1 or TMEM236 signaling.

For example, the SERPINB1 or TMEM236 inhibiting agent can be an anti-SERPINB1 or anti-TMEM236 antibody. Furthermore, the anti-SERPINB1 or anti-TMEM236 antibody can be a murine antibody, a humanized murine antibody, or a human antibody.

As another example, the SERPINB1 or TMEM236 inhibiting agent can be an anti-SERPINB1 or anti-TMEM236 antibody, wherein the anti-SERPINB1 or anti-TMEM236 antibody prevents binding of SERPINB1 or TMEM236 to its receptor, or prevents activation of SERPINB1 or TMEM236 and downstream signaling.

As another example, the SERPINB1 or TMEM236 inhibiting agent can be a fusion protein. For example, the fusion protein can be a decoy receptor for SERPINB1 or TMEM236, Furthermore, the fusion protein can comprise a mouse or human Fc antibody domain fused to the ectodomain of SERPINB1 or TMEM236.

As another example, a SERPINB1 or TMEM236 inhibiting agent can be an inhibitory protein that antagonizes SERPINB1 or TMEM236. For example, the SERPINB1 or TMEM236 inhibiting agent can be a viral protein, which has been shown to antagonize SERPINB1 or TMEM236.

As another example, a SERPINB1 or TMEM236 inhibiting agent can be a short hairpin RNA (shRNA) or a short interfering RNA (siRNA) targeting SERPINB1 or TMEM236.

As another example, a SERPINB1 or TMEM236 inhibiting agent can be an sgRNA targeting SERPINB1 or TMEM236R1.

Methods for preparing a SERPINB1 or TMEM236 inhibiting agent (e.g., an agent capable of inhibiting SERPINB1 or TMEM236 signaling) can comprise construction of a protein/Ab scaffold containing the natural SERPINB1 or TMEM236 receptor as a SERPINB1 or TMEM236 neutralizing agent; developing inhibitors of the SERPINB1 or TMEM236 receptor “down-stream”; or developing inhibitors of the SERPINB1 or TMEM236 production “up-stream”.

Inhibiting SERPINB1 or TMEM236 can be performed by genetically modifying SERPINB1 or TMEM236 in a subject or genetically modifying a subject to reduce or prevent expression of the SERPINB1 or TMEM236 gene, such as through the use of CRISPR-Cas9 or analogous technologies, wherein, such modification reduces or prevents a disease.

### Chemical Agent:

Examples of vaccine modulation agents are described herein. R groups can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above, and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include-lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na+])+0.41 (fraction G/C content)−0.63 (% formamide)−(600/1). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, SERPINB1, TMEM236, and G1P signals can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of SERPINB1 or TMEM236 by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for a vaccine modulation agent to target cells by the removal of SERPINB1 or TMEM236 signals.

For example, the methods as described herein can comprise a method for altering a target polynucleotide sequence in a cell comprising contacting the polynucleotide sequence with a clustered regularly interspaced short palindromic repeats-associated (Cas) protein.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing a viral disease in a subject in need of administration of a therapeutically effective amount of a vaccine, so as to inoculate a subject of a viral disease.

Methods described herein are generally performed on a subject in need thereof. A subject in need of the therapeutic methods described herein can be a subject having, diagnosed with, suspected of having, or at risk for developing a viral disease. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a vaccine is, for example, an amount that would cause the desired therapeutic effect in a subject while minimizing undesired side effects. In various embodiments, an effective amount of a vaccine described herein can substantially inhibit a viral infection, slow the progress of a viral disease, or limit the development of a viral disease.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a vaccine can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat a viral infection.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a vaccine can occur as a single event or over a time course of treatment. For example, a vaccine can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a viral infection.

A vaccine can be administered simultaneously or sequentially with another agent, such as an antibiotic, an anti-inflammatory, or another agent. For example, a vaccine can be administered simultaneously with another agent, such as an antibiotic or an anti-inflammatory. Simultaneous administration can occur through administration of separate compositions, each containing one or more of a vaccine, an antibiotic, an anti-inflammatory, or another agent. Simultaneous administration can occur through administration of one composition containing two or more of a vaccine, an antibiotic, an anti-inflammatory, or another agent. A vaccine can be administered sequentially with an antibiotic, an anti-inflammatory, or another agent. For example, a vaccine can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: Chem Bridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about-2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include but are not limited to Vero cells and cell culture materials. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Genome-Wide Screens Identify Factors that Improve Rotavirus Reverse Genetics and Vaccine Production

Rotaviruses pose a significant threat to the health of young children. To identify host factors that modulate rotavirus infection, genome-wide loss-of-function CRISPR/Cas9 screens using rhesus rotavirus RRV strain and a matched African green monkey library were performed. Genetic deletion of SERPINB1 or TMEM236, the top anti-viral factors, increased virus titers and plaque sizes in a rotavirus strain-independent manner. The existing rotavirus reverse genetics systems was optimized by combining SERPINB1 knockout cells with a helper plasmid, which ensures autonomous synthesis of the viral mRNA with key post-transcriptional modifications. Virus recovery efficiency was improved and several low-titer rotavirus reporter and mutant strains that are otherwise difficult to rescue were rescued. Furthermore, it was demonstrated that Vero cells with TMEM236 disruption supported higher yields of two live-attenuated rotavirus vaccine strains than the parental cell line and thus represent a more robust vaccine-producing cell substrate. Collectively, the antiviral factors identified in this study facilitated the development of a third-generation optimized rotavirus reverse genetics system as well as gene-edited Vero cells to improve vaccine production.

### Introduction

Rotaviruses (RVs) belong to the Sedoreoviridae family, a group of double-stranded RNA (dsRNA) non-enveloped viruses. The genome of RV consists of 11 segments (genes 1-11), which encode 6 structural proteins (VP1-4, VP6, and VP7) and 6 nonstructural proteins (NSP1-6). Although there are two vaccines available globally, i.e., Rotarix® (RV1, GSK), and RotaTeq® (RV5, Merck), RV infection results in the death of more than 128,500 children per year worldwide, highlighting the need for more efficacious vaccines.

The first reverse genetics system for RV was described in 2006, in which a human RV strain was used as a helper virus. The transformative plasmid-only-based reverse genetics system for RV was developed in 2017. In this system, each of 11 RV cDNAs is driven by a T7 promoter and ends with a hepatitis delta virus ribozyme sequence. The 11 rescue plasmids are transfected into BHK-T7 cells along with plasmids encoding two subunits of the vaccinia virus capping enzyme (D1R and D12L) and a Nelson Bay reovirus fusion-associated small transmembrane protein (FAST). Because RV does not replicate efficiently in BHK-T7 cells, MA104 cells, which are highly susceptible to RV infection, are used as an overlay to propagate the rescued virus. The lab previously optimized this system by increasing the amount of the rescue plasmids of NSP2 and NSP5 proteins, components of viroplasm where RV replication occurs. Also, replaced were the plasmids encoding vaccinia virus capping enzymes with a single plasmid C3P3-G1 (i.e. first generation of cytoplasmic chimeric capping-prone phage polymerase system) that encodes a fusion protein of African swine fever virus capping enzyme and T7 polymerase. Furthermore, the wild-type MA104 cells were replaced with MA104 N*V cells constitutively expressing bovine viral diarrhea virus N protein and parainfluenza virus 5 V protein that degrade IRF3 and STAT1, respectively. Subsequently, our lab and others have optimized the RV reverse genetics system to enable the recovery of low-titer recombinant reporter viruses and hard-to-rescue RV strains, including simian RV, human RV, porcine RV, and murine-like RV. However, the limited efficiency in recovering these hard-to-rescue strains reveals a critical gap for RV reverse genetics systems. Multiple potential means to increase rescue efficiency have been recently discussed, one of which is an optimized MA104 cell substrate for the overlaying step.

Whole genome loss-of-function CRISPR/Cas9 screens have been used to identify pro-viral host factors for RV, influenza, SARS-CoV-2, hepatitis B virus, hepatitis C virus, flavivirus, and murine norovirus, but fewer studies have focused on the anti-viral host factors. Here, the full-genome loss-of-function CRISPR/Cas9 survival was leveraged, as well as cell sorting-based screens to identify both pro-viral and anti-viral host factors for RV in MA104 cells. Based on results stemming from this screening, the SERPINB1 knockout cells were used to create a new RV reverse genetics system, which enables the rescue of difficult-to-recover viruses. It was also found that TMEM236 knockout cells support higher levels of viral replication and are thus a compelling candidate cell line for RV and other vaccine production.

### Results

### a Fluorescence-Activated Cell Sorting (FACS)-Based CRISPR Screen Identified Anti-Rotavirus Host Factors.

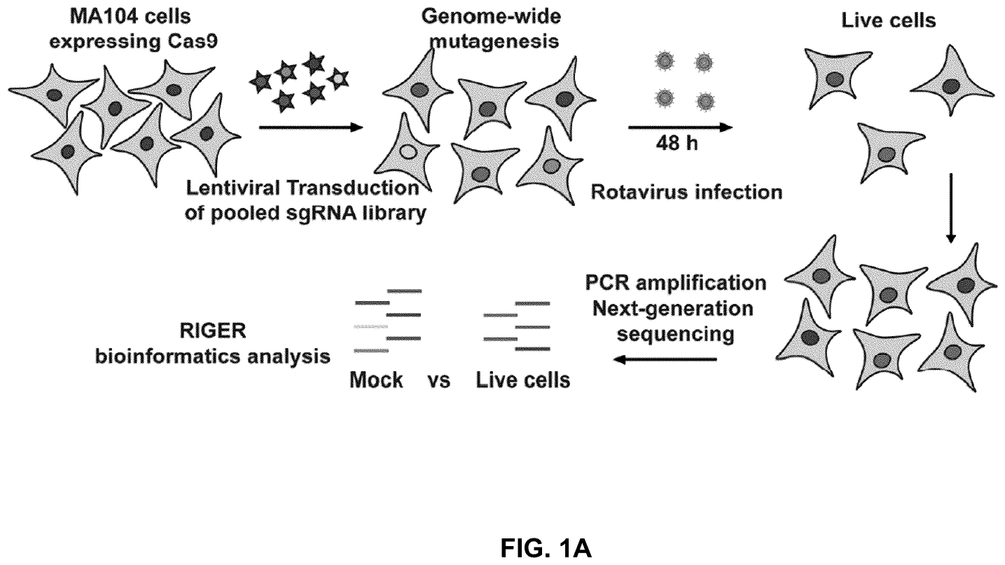

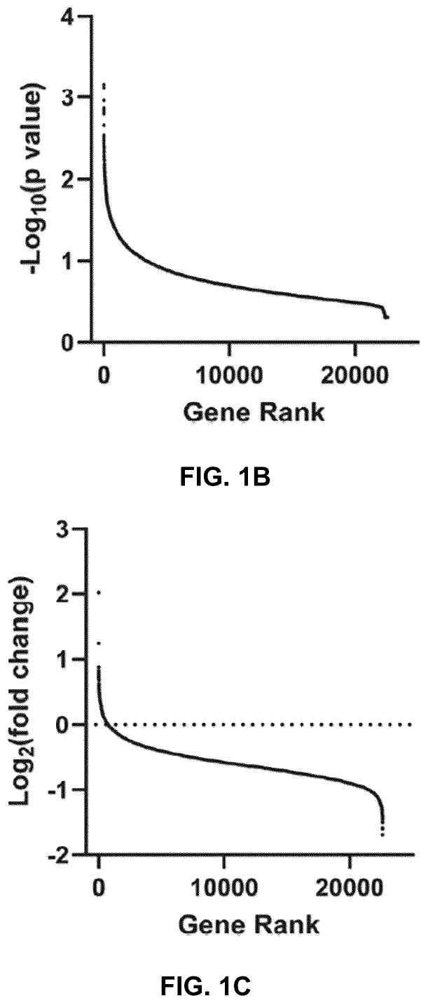

To permit genome-wide CRISPR/Cas9 screening for RV host dependency factors, first the lentivirus was packaged expressing Cas9, transduced MA104 cells, and selected a high-performance clone (MA104-Cas9 cells). Cells were then transduced with C. sabaeus genome-wide pooled CRISPR library and selected with puromycin. Subsequently, the MA104 library cells were randomly divided into 4 groups, with 2 groups for mock infection and 2 groups challenged with simian RV RRV strain. Cells that were resistant to RRV-induced cytopathic effects were harvested and then amplified for sgRNA sequencing (FIG. 1A). The RNA interference Gene Enrichment Ranking (RIGER) algorithm was used to rank enriched genes, taking into account multiple different sgRNAs per gene, number of sequencing reads per gene, and the enrichment of sgRNAs compared to the uninfected pooled library. A large panel of host-dependency factors for RV infection were identified (FIG. 1B). However, the overall average fold change of the screen was less than 2-fold (FIG. 1C), suggesting that a better screening strategy was needed.

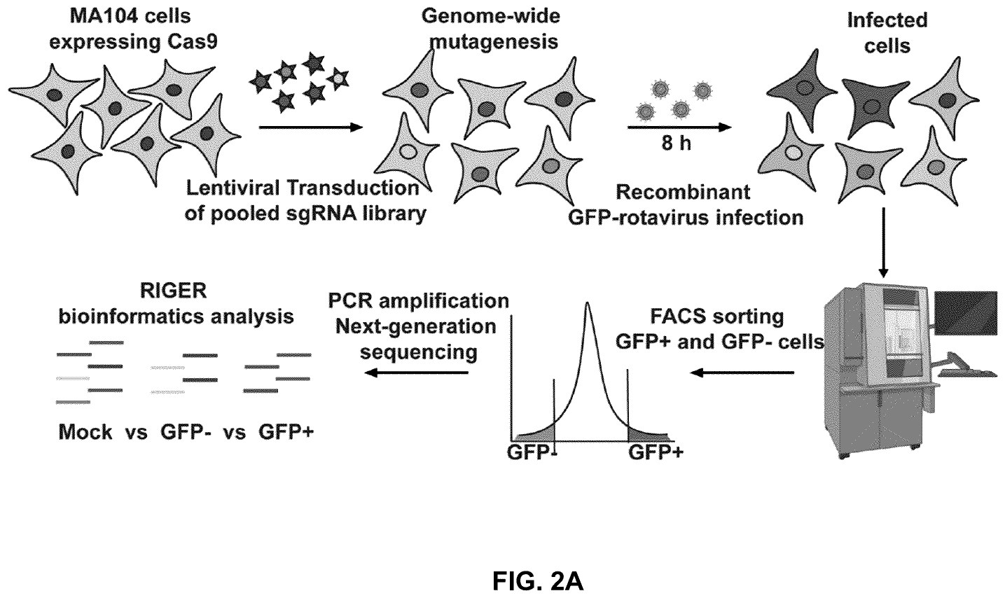

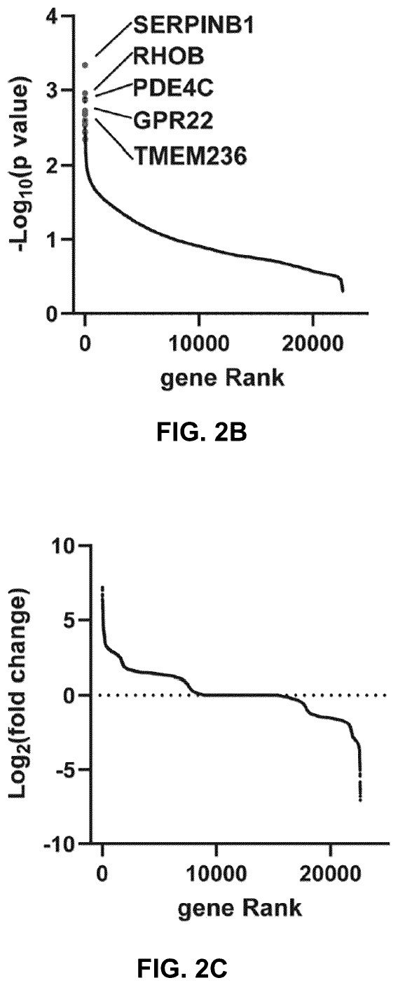

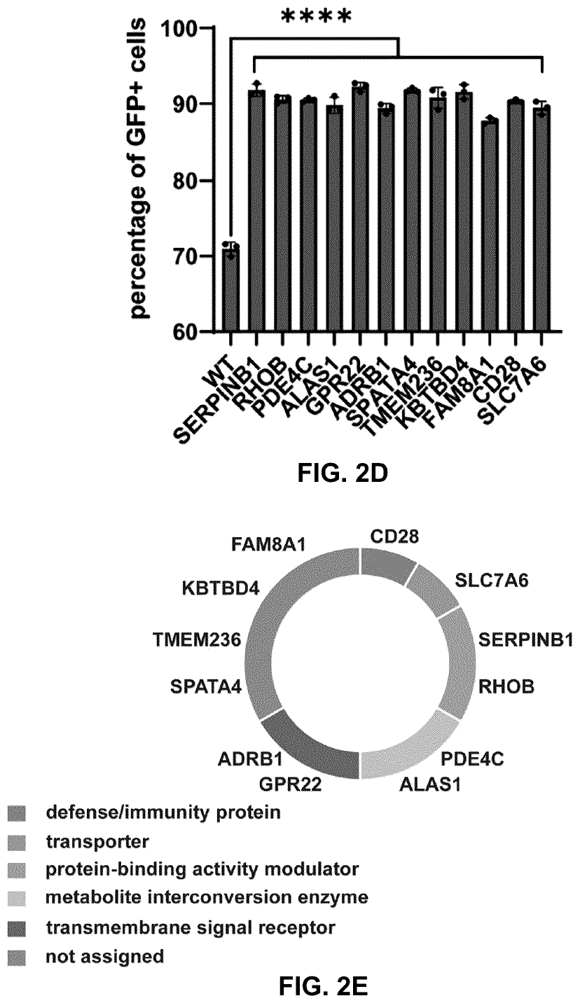

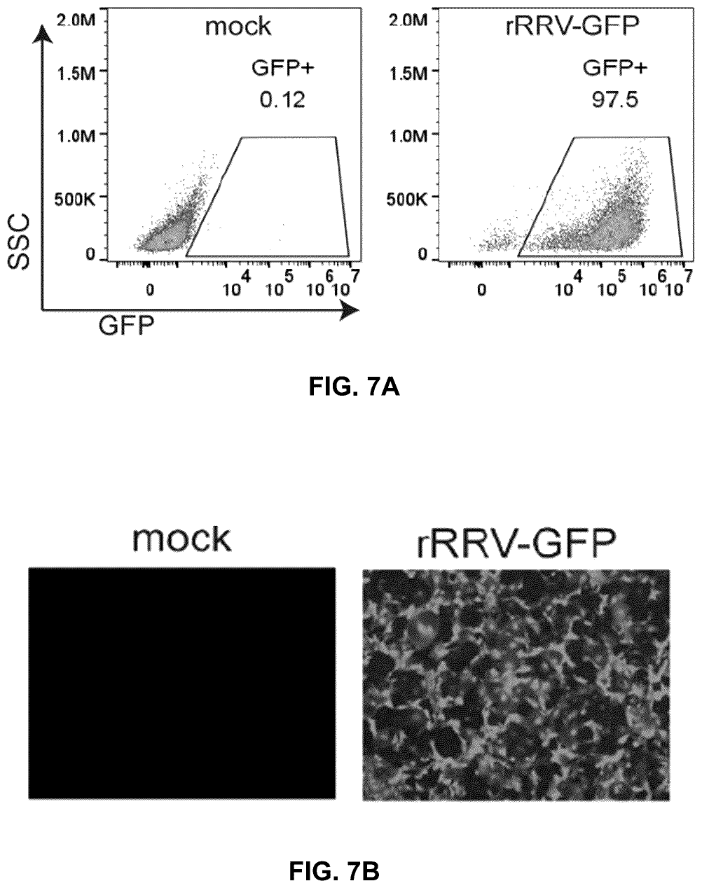

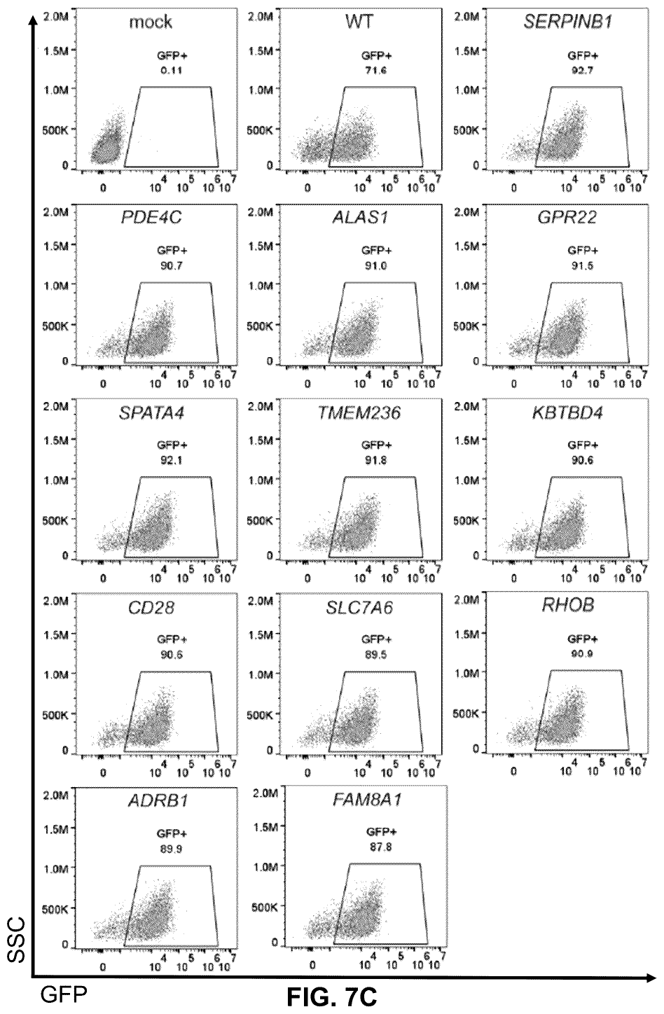

Next a FACS-based screen was performed for both pro-RV and anti-RV host factors. To achieve our goal, we made a high titer stock of a recombinant RRV that encodes a GFP reporter (rRRV-GFP), and optimized conditions so that more than 97% of the parental MA104-Cas9 cells were infected based on flow cytometric analysis and microscopy (FIG. 7A, FIG. 7B). Using the GFP fluorescence intensity as a surrogate for infectivity, the top 0.1% of dimmest cells and brightest cells were sorted, which represent pro-viral factors and anti-viral factors, respectively, of the MA104 library at a single cycle of RV replication, i.e., 8 h post infection (hpi) (FIG. 2A). Next, RIGER was used to identify enriched sgRNAs in sorted cells. A total of 103 anti-viral genes were identified with the cutoff of −log(p value)>1.7, sgRNA hits≥4, and 74 pro-viral genes were identified with the cutoff of −log (p value)>1.7, sgRNA hits≥4. To further investigate enriched gene functions during RV infection, the top 12 anti-viral hits were selected for subsequent studies based on their enrichment between GFP positive and GFP negative cells: SERPINB1, RHOB, PDE4C, GPR22, ADRB1, FAM8A1, KBTBD4, TMEM236, CD28, SLC7A6, ALAS1, and SPATA4 (FIG. 2B, FIG. 2C). Individual gene knockout MA104 cells were generated by lenti-CRISPR_v2-based lentiviral transduction with the highest scoring sgRNA (Table 1) and puromycin selection. As expected, knockout of these genes significantly increased virus infectivity at 8 hpi (FIG. 2D, FIG. 7C). These results indicated the successful identification of genes that have anti-RV activities by using a FACS-based screen. Next, a gene ontology pathway analysis was performed, and results showed that SERPINB1 and RHOB are protein-binding activity modulators; PDE4C and ALAS1 are metabolite interconversion enzymes; GPR22 and ADRB1 are transmembrane signal receptors; CD28 is a defense/immunity protein; SLC7A6 is a transporter; FAM8A1, KBTBD4, TMEM236, and SPATA4 do not yet have well-defined functions (FIG. 2E). Overall, membrane-associated proteins were overrepresented and consistent with our experimental design of a single round of infection.

### Genetic Deletion of SERPINB1 or TMEM236 Genes Enhanced the Infectivity of Multiple RV Strains.

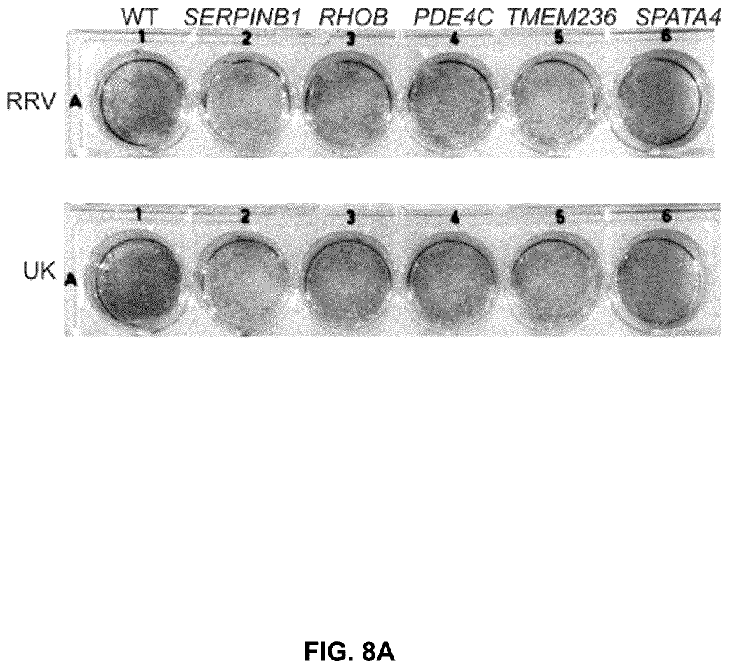

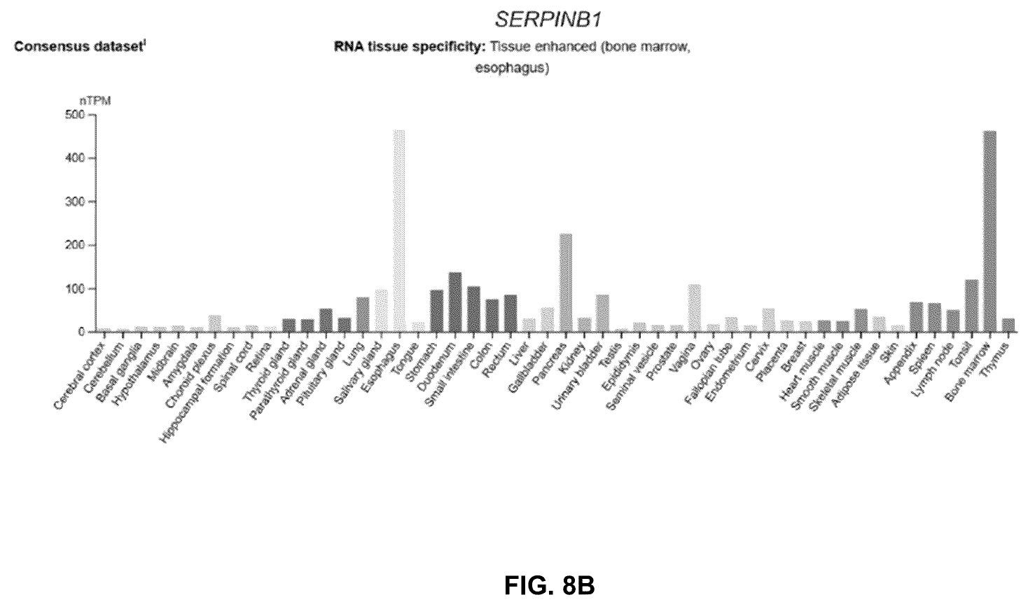

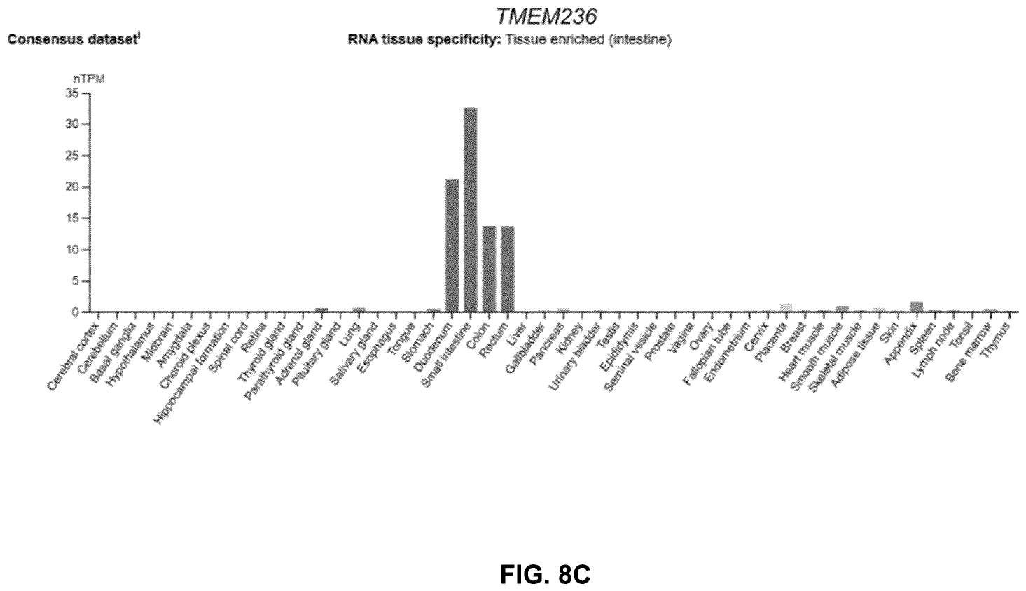

To narrow down targets for in-depth characterization, RT-qPCR-based viral RNA quantification was performed and the percentage of GFP positive cells at 24 hpi was measured to determine viral antigen levels in the infected MA104 cells. SERPINB1, RHOB, PDE4C, TMEM236, and SPATA4 pooled knockout MA104 cells had the highest RV RNA and protein levels and were selected for further studies. Crystal violet staining was used to evaluated the cytopathic effect following infection by rhesus RV RRV strain or bovine RV UK strain in different gene knockout MA104 cells at 24 hpi (FIG. 8A). Loss of SERPINB1 or TMEM236 accelerated cell death from RV infection. Given that RV has a tissue tropism for the small intestine, the lab assessed the RNA levels of SERPINB1 and TMEM236 in publicly available Human Protein Atlas dataset. Interestingly, both SERPINB1 and TMEM236 are expressed at high levels in the human small intestine (FIG. 8B, FIG. 8C). Thus, SERPINB1 and TMEM236 were focused on for functional analyses.

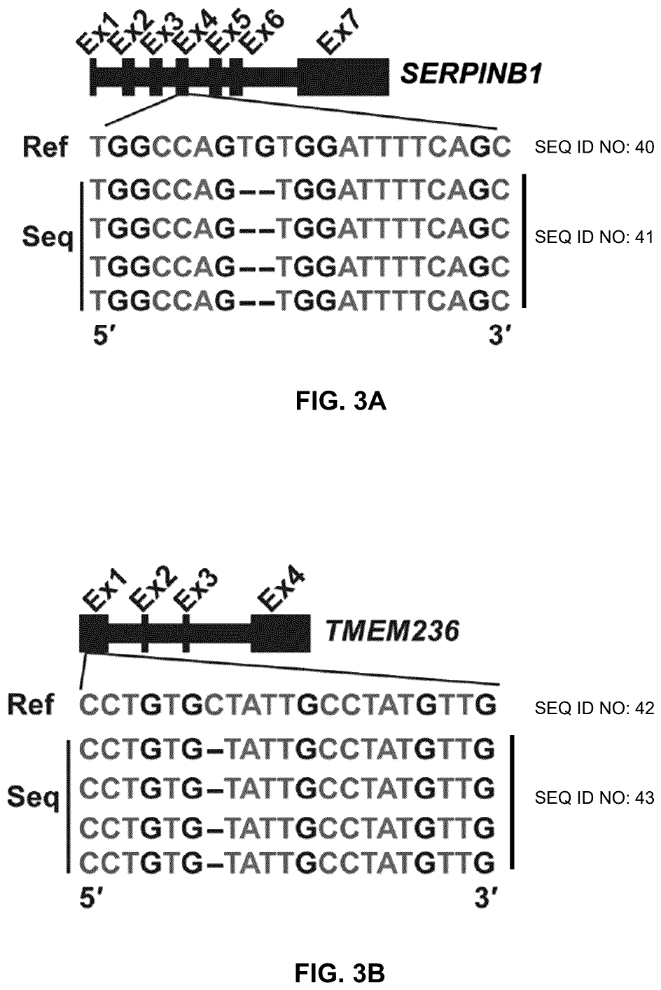

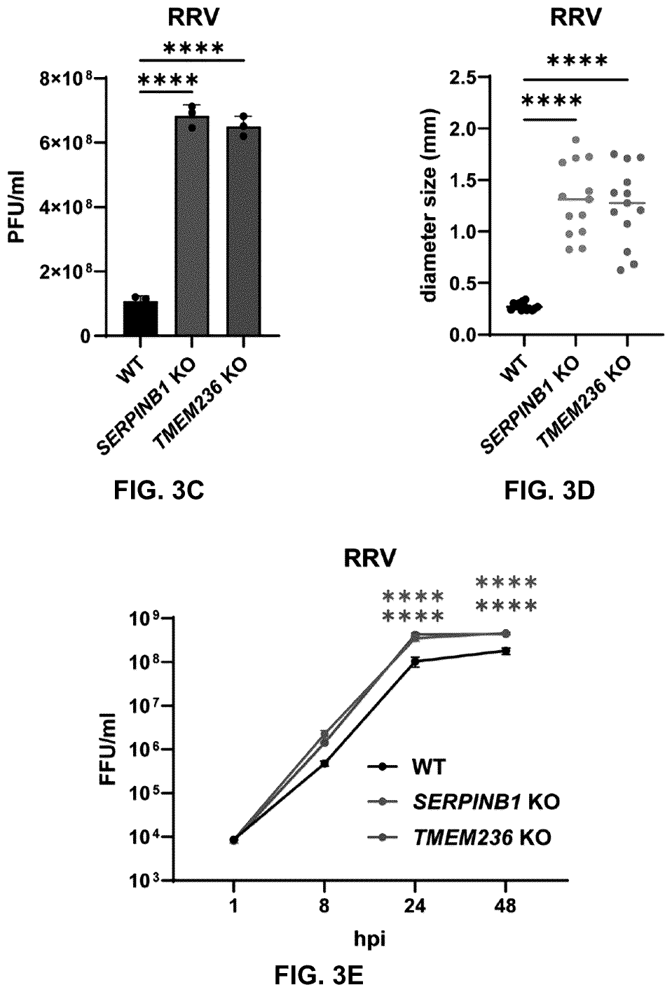

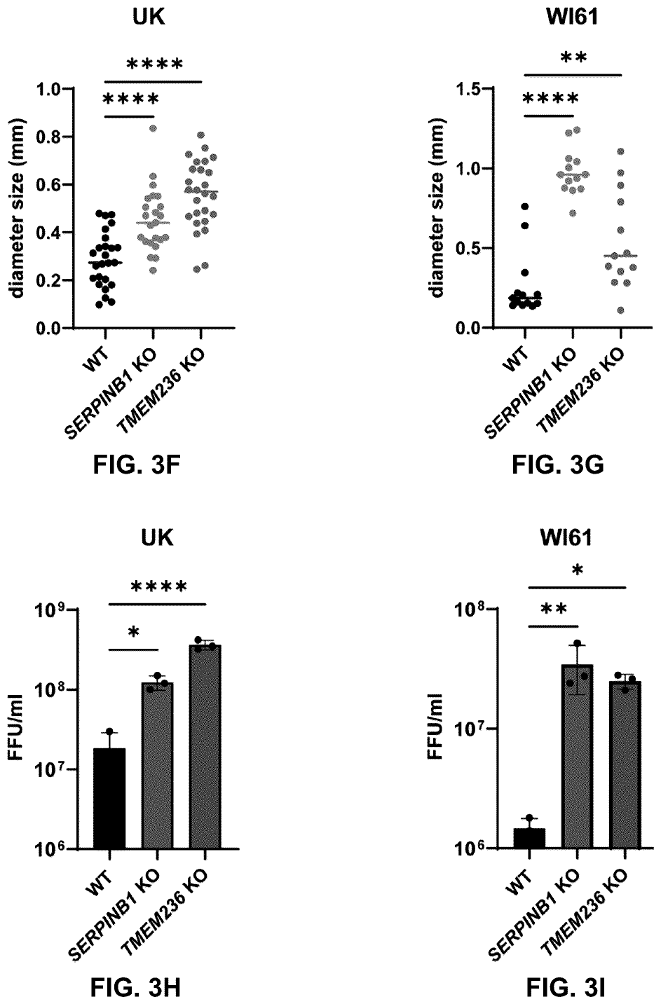

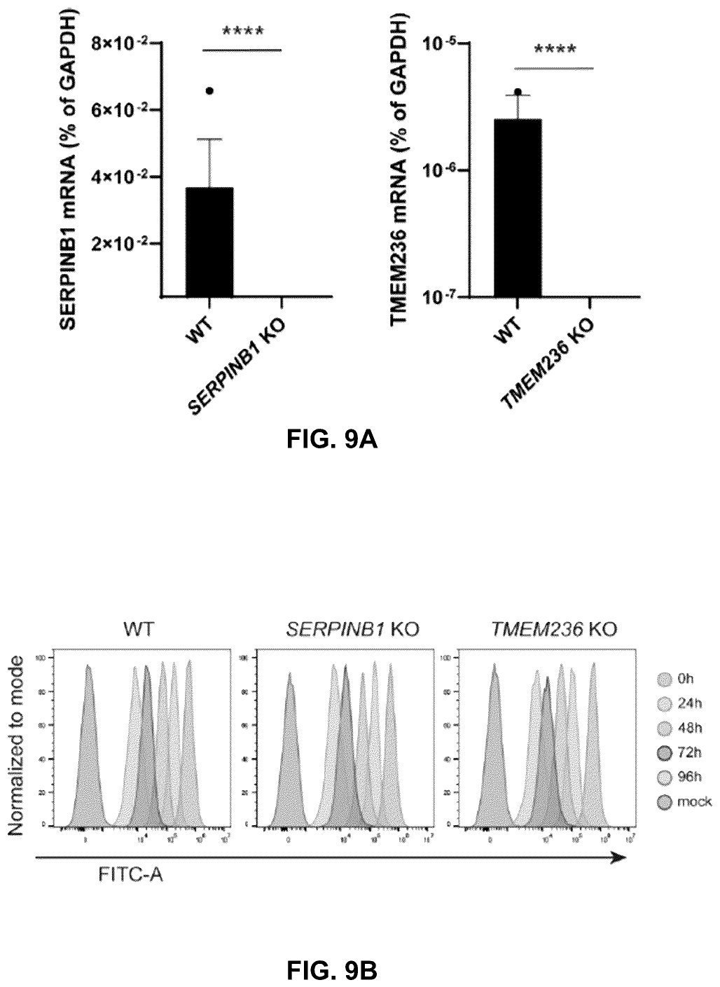

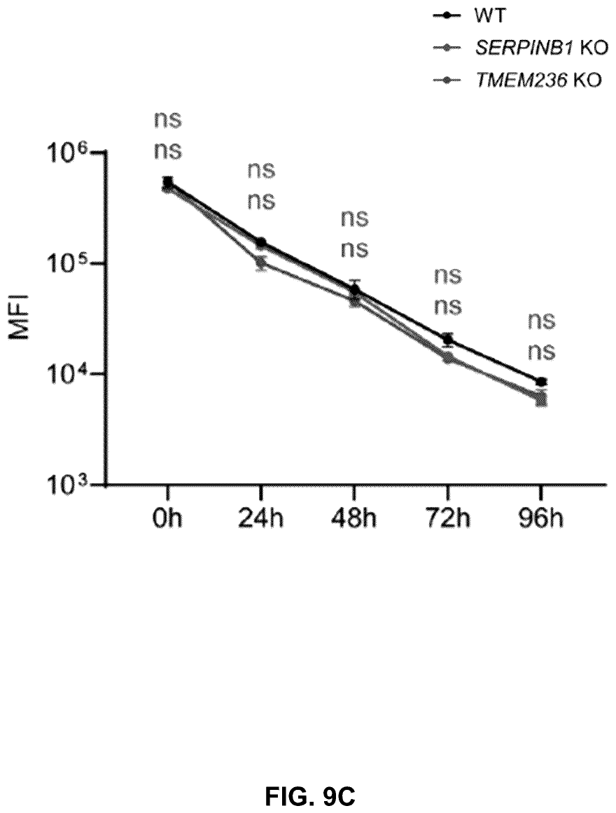

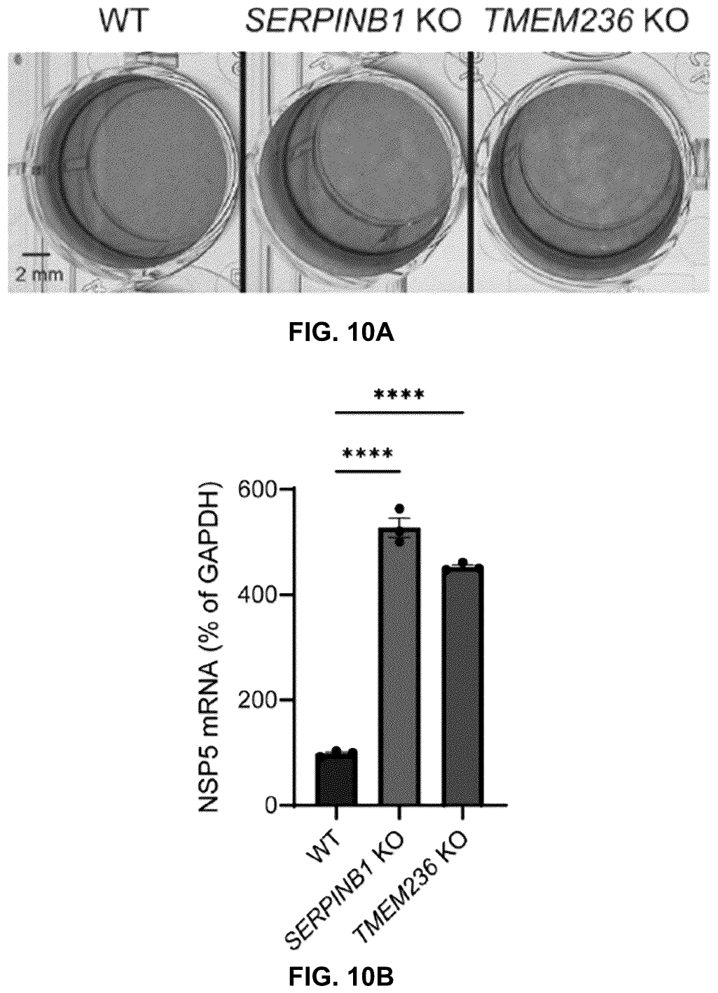

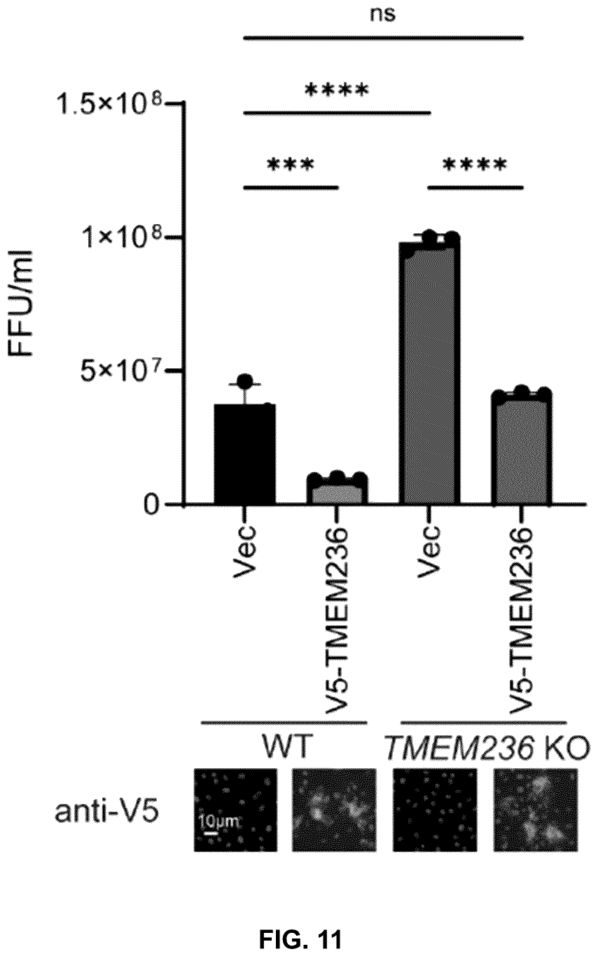

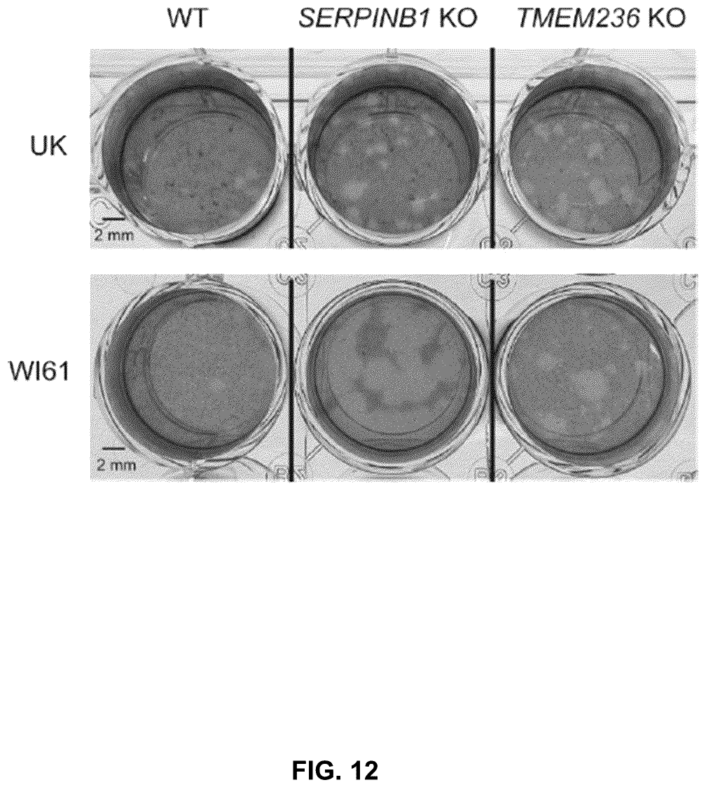

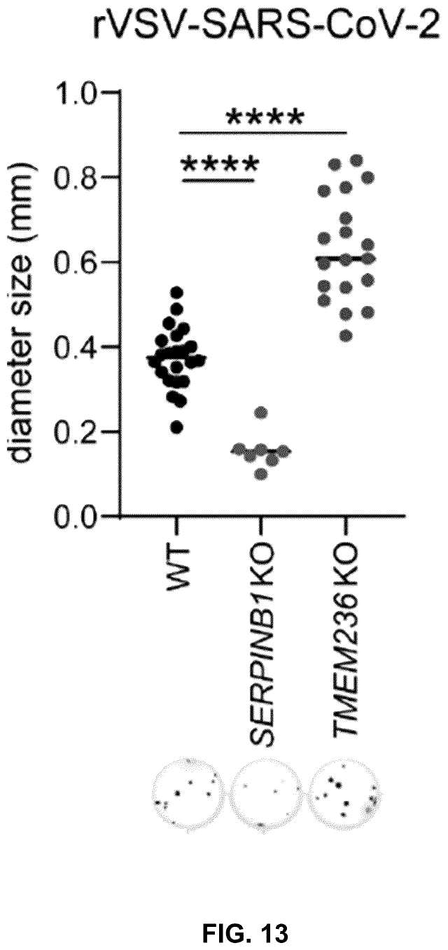

Single clonal SERPINB1 or TMEM236 knockout MA104 cell lines were generated with the highest scoring sgRNAs. Due to the lack of validated antibodies to detect monkey proteins by western blot, clean knockout cells were verified by Sanger sequencing (FIG. 3A, FIG. 3B) and qRT-PCR primers that target the last exons of SERPINB1 and TMEM236 genes (FIG. 9A). The growth rate of SERPINB1 and TMEM236 knockout cells was comparable to that of the wild-type cells (FIG. 9B, FIG. 9C). Subsequently, it was verified that RRV replication was significantly increased in SERPINB1 or TMEM236 knockout cell lines (FIG. 3C) and that plaque size was enhanced (FIG. 3D, FIG. 10A). Intracellular viral RNA levels were also increased in knockout cells (FIG. 10B). RRV infection was inhibited in TMEM236-overexpressing MA104 cells (FIG. 11). Investigated next was the replication kinetics of RRV in vitro. An increase in virus titer in knockout cells was seen as early as 8 hpi, and significantly increased at later time points, i.e., 24, and 48 hpi (FIG. 3E). To explore whether the role of these genes is specific to RRV, the analysis was expanded to the bovine RV UK and human RV WI61 strains. Disruption of SERPINB1 or TMEM236 was associated with notably increased plaque sizes (FIG. 3F, FIG. 3G) and virus titers for both viral strains (FIG. 3H, FIG. 3I, FIG. 12). Given that MA104 cells have endogenous monkey ACE2 that supports SARS-CoV-2 infection, further assessed was the effect of SERPINB1 and TMEM236 on a recombinant vesicular stomatitis virus encoding the spike protein of SARS-CoV-2 (rVSV-SARS-CoV-2) infection. It was found that while TMEM236 knockout increased rVSV-SARS-CoV-2 plaque size, and SERPINB1 knockout inhibited plaque formation (FIG. 13). These results suggest that TMEM236 may broadly inhibit virus infection while the anti-viral effect of SERPINB1 is more specific to RV infection.

The Efficiency of RV Reverse Genetics Systems was Improved by Overlaying with the SERPINB1 Knockout MA104 Cells and the Use of a C3P3-G3 Plasmid

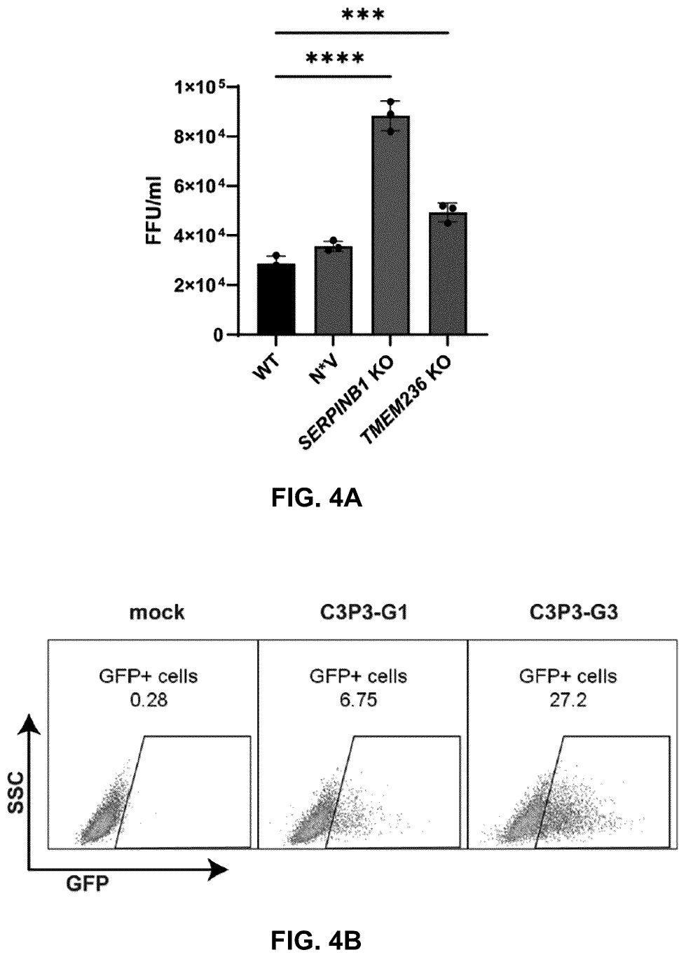

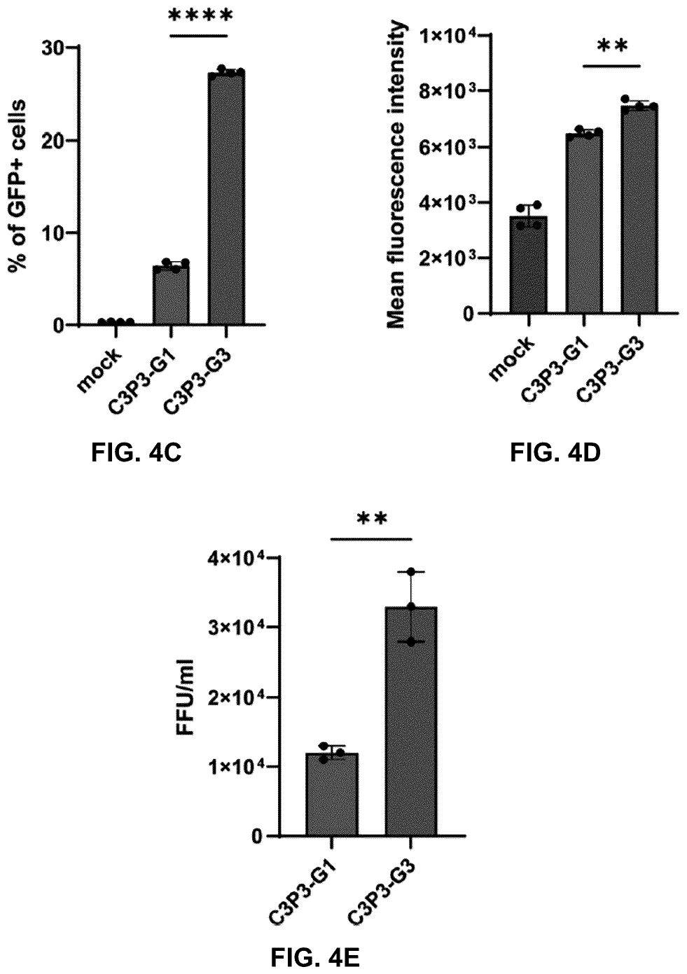

The translational utility of RV as an enteric viral vector is hampered by the inability to efficiently rescue reporter and low-titer RV strains, highlighting the need for a better reverse genetics system. Toward this goal, it was investigated whether the rescue efficiency could be improved by overlaying transfected BHK-T7 cells with genetically modified SERPINB1 or TMEM236 knockout cells instead of wild-type MA104 cells and MA104 N*V cells. As shown in FIG. 4A, the production of infectious virus particles of the prototypic simian RV SA11 strain was increased by 4-fold in BHK-T7 cell lysates after overlaying with SERPINB1 knockout cells. Another way to increase the rescue efficiency is to supply more T7 DNA-dependent RNA polymerase. Use of C3P3-G1, a fusion protein of African swine fever virus capping enzyme NP868 and T7 RNA polymerase which generates viral mRNAs having ^{m7}G-cap at their 5′-ends, has been shown to optimize the original reverse genetics system and improve efficiency. A recent study showed that the C3P3-G2 system brings an engineered cytoplasmic PAPα to the target mRNA to elongate its poly(A) tail, which leads to an increase in protein expression levels of about 2.5-3 times in cultured human cells compared to the C3P3-G1 system. Detailed characterization of the third-generation C3P3-G3 system will be published separately. C3P3-G1 or C3P3-G3 were co-transfected with a pT7 plasmid encoding RRV-NSP3-GFP, which was used for reporter virus generation. After 24 hpi, GFP signals were measured by flow cytometry analysis (FIG. 4B). Both the percentage of GFP positive cells and the mean fluorescence intensity was increased by 5-fold in C3P3-G3 prototype transfected cells as compared to the original C3P3-G1 system (FIG. 4C, FIG. 4D). To investigate whether the inclusion of a C3P3-G3 prototype plasmid produces more viral particles, SA11 virus was rescued using the C3P3-G3 helper plasmid. Indeed, observed was a 3-fold increase in the yield of infectious virus particles in C3P3-G3 transfection compared to C3P3-G1 (FIG. 4E), suggesting that C3P3-G3 seems to outperform C3P3-G1.

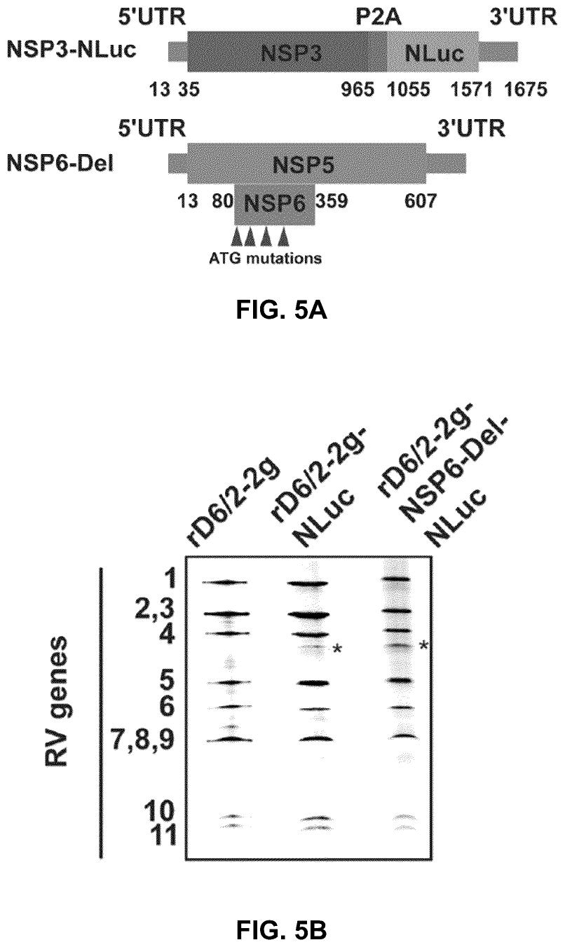

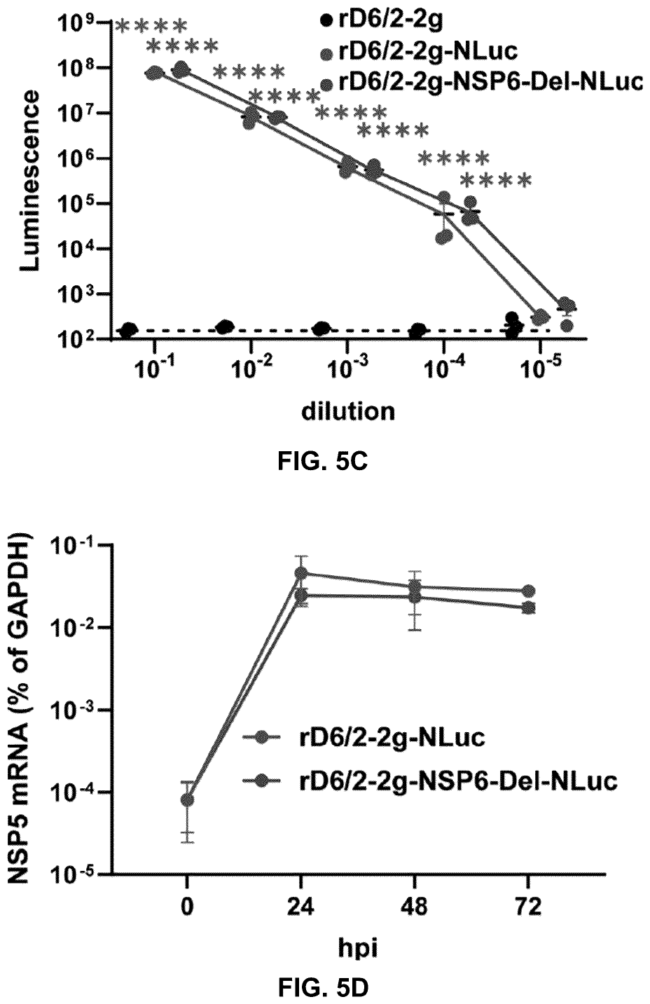

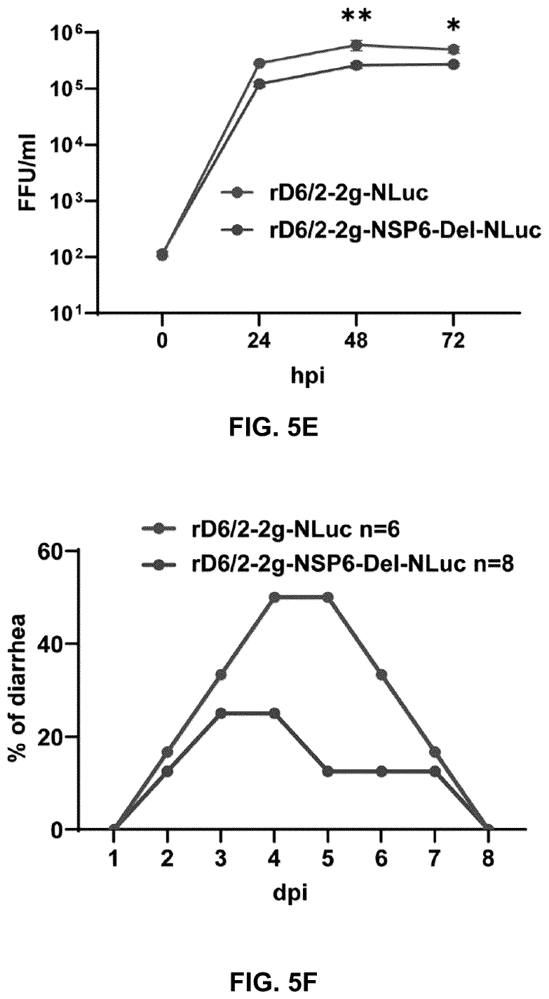

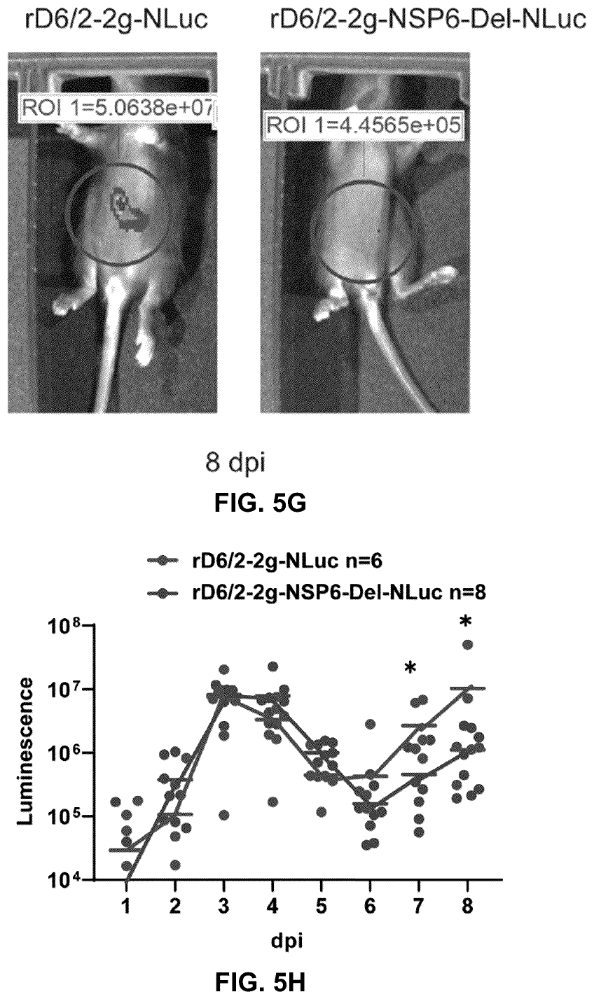

Next, to assess whether SERPINB1 knockout cells are better than MA104 N*V cells, the recovery efficiency was compared using a fixed C3P3-G1 system. The lab was able to rescue challenging viruses which cannot be rescued with the previous already optimized system, for example, a murine-like rD6/2-2 g strain with all the possible start codons of NSP6 removed and expressing a Nano-luciferase reporter (NLuc) in the NSP3 gene segment, abbreviated as rD6/2-2g-NSP6-Del-NLuc (FIG. 5A). Also achieved was the successful rescue of a highly attenuated recombinant virus rSA11-RotaTeq-VP4 (human RV VP4) with an SA11 backbone encoding the VP4 gene derived from the RotaTeq vaccine strain. Because both overlaying BHK-T7 cells with SERPINB1 knockout MA104 cells and using a C3P3-G3 plasmid increased the yield of RV by different mechanisms, these two methods were combined to create a new reverse genetics system. The lab directly compared the rescue efficiency of SERPINB1 knockout in combination with C3P3-G3 with the previously published methods. Consistent with prior data, our system is comparable (above 80% success rate) to Sanchez-Tacuba's method in terms of rescuing rRRV and rD6/2-2 g, a murine-like RV strain. In addition, our system allowed us to increase the rescue efficiency of rD6/2-2g-NSP6-Del-NLuc and rSA11-RotaTeq-VP4.

### NSP6 Promoted the Replication and Virulence of Murine RV In Vivo

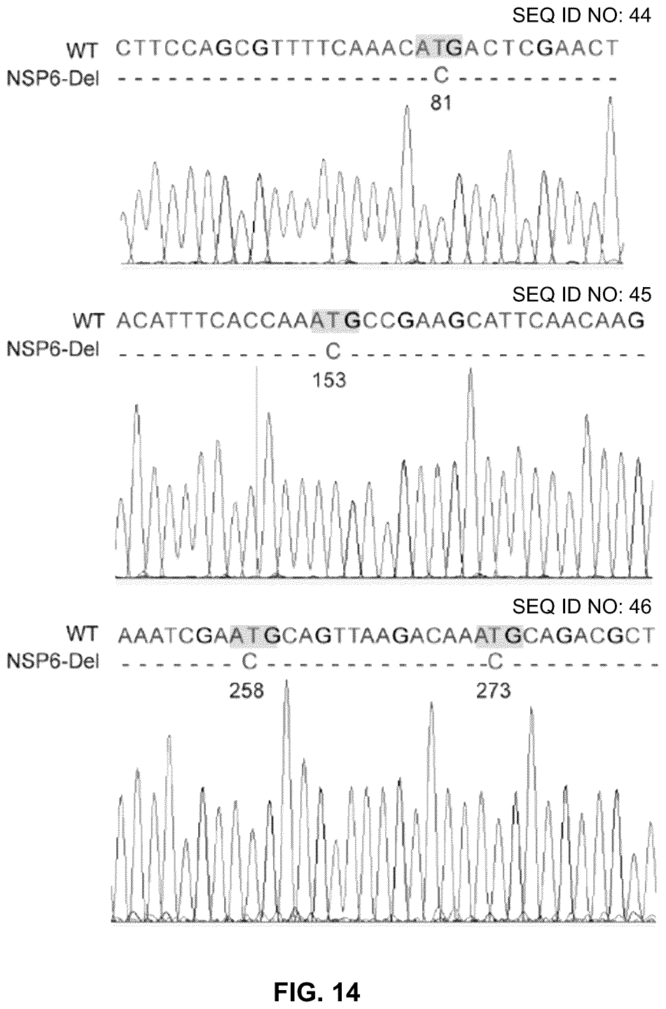

NSP6 is the least characterized RV protein. It is only 12 kDa and some RV strains do not encode NSP6. Previous studies have shown that NSP6 deletion in the simian SA11 background does not impact viral replication in vitro or pathogenesis in a mouse model of infection. However, SA11 is a simian RV and has only limited replication capacity in mice, rendering determination of NSP6's function in vivo inconclusive. Our previous work revealed that the murine-like RV, rD6/2-2 g, encoding an NLuc reporter, has all the properties of bona fide murine RVs, including fecal shedding, diarrhea development, and transmission to uninfected littermates in the same cage. The optimized reverse genetics system was leveraged to rescue the rD6/2-2g-NSP6-Del-NLuc virus as described above. The mutations of start codons in NSP6 were confirmed in the rescued virus stocks by Sanger sequencing (FIG. 14). The identity of rD6/2-2g-NSP6-Del-NLuc was validated by a unique electropherotype by RNA polyacrylamide gel electrophoresis analysis (FIG. 5B) and Sanger sequencing. The edited dsRNA of RV gene segment 7 migrated similarly to rD6/2-2g-NLuc but slower than the rD6/2-2 g gene segment 7 due to the NLuc insertion (FIG. 5B). The luciferase activity in RV-infected cells was not affected by the introduction of NSP6 deletion (FIG. 5C). In addition, it was determined that the replication kinetics of rD6/2-2g-NSP6-Del-NLuc compared to the parental rD6/2-2g-NLuc in vitro. The intracellular mRNA levels (FIG. 5D) and virus titers (FIG. 5E) were similar to rD6/2-2g-NLuc at 24, 48, and 72 hpi, suggesting that NSP6 is not essential for RV replication in vitro, consistent with prior work. Investigated next, was the role of NSP6 in vivo. Five-day-old C57BL/6 mice were inoculated with a low inoculum (3.5×10³ FFUs) of rD6/2-2g-NLuc and rD6/2-2g-NSP6-Del-NLuc via oral gavage. It was observed that about 10% of mice developed diarrhea at 2 days post infection (dpi) about 25% developed diarrhea of rD6/2-2g-NSP6-Del-NLuc virus at 3 dpi. Despite a small number of animals, the percentage of mice exhibiting diarrhea was decreased in the NSP6 deletion virus-infected animals (FIG. 5F). To measure tissue viral loads, the bioluminescence signals from days 0 to 8 post infection were quantified and observed strong luciferase in the abdominal cavity as early as 1 dpi using the in vivo imaging system, with a significant decrease at 7 and 8 dpi in the absence of NSP6 (FIG. 5G, FIG. 5H). Taken together, these data suggest that while NSP6 is not required for murine RV replication in vitro, it may be important for RV pathogenesis in vivo. These findings further serve as a proof-of-principle of the prowess of our new reverse genetics system.

### TMEM236 Knockout Vero Cells Supported Increased Production of Live-Attenuated RV Vaccines.

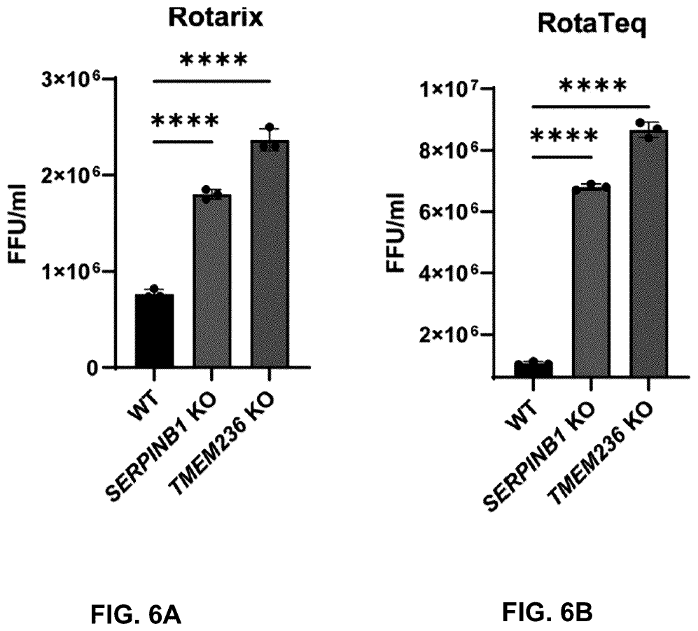

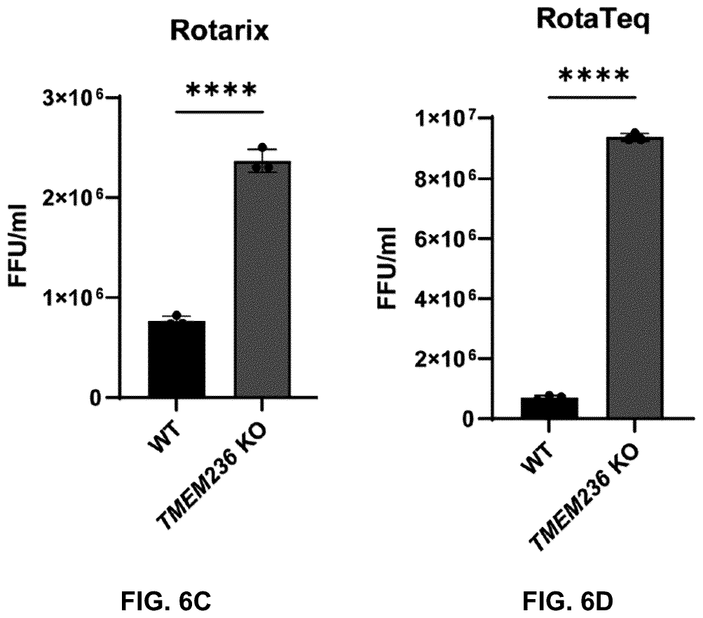

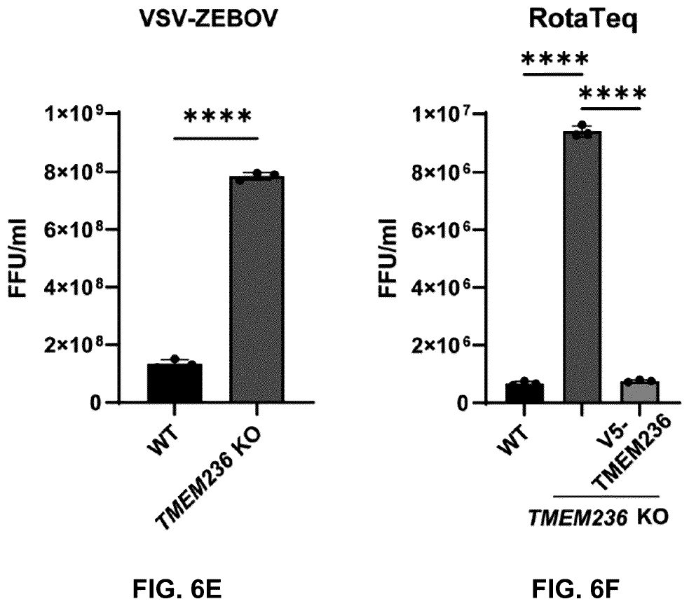

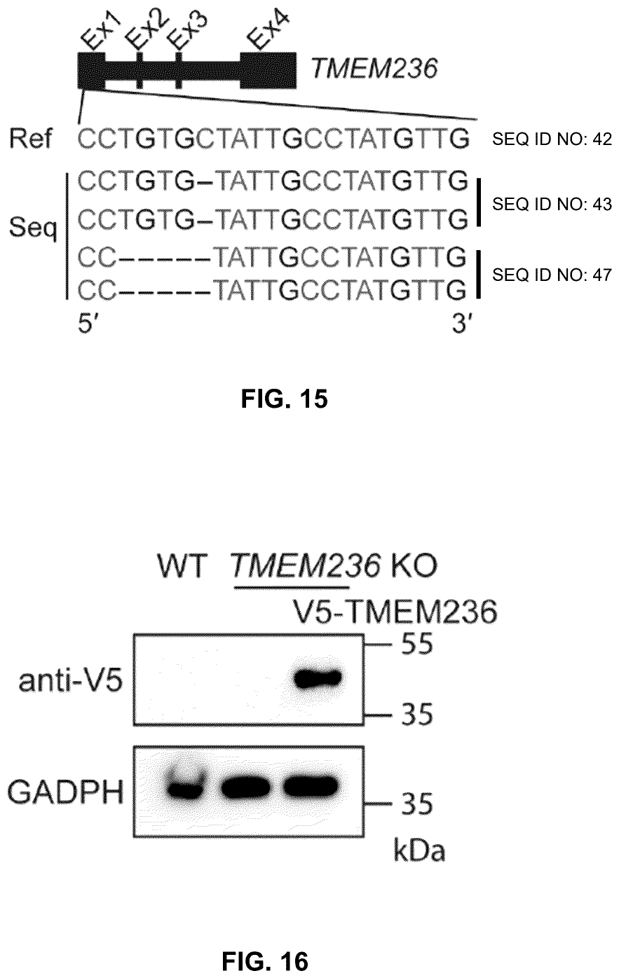

The very first RV vaccine Rotashield is based on RRV and was approved by the FDA in 1998. Because the data shows increased RRV replication in anti-viral gene knockout MA104 cells (FIG. 3C, FIG. 3D, FIG. 3E), the analysis was extended to the currently two live attenuated vaccines Rotarix and RotaTeq administered worldwide. To investigate whether SERPINB1 and TMEM236 have effects on vaccine strain propagation, the wild type, SERPIBN1, and TMEM236 knockout MA104 cells were infected with either Rotarix or RotaTeq. It was found that both SERPINB1 and TMEM236 knockout supported statistically significantly increased virus titers (FIG. 6A, FIG. 6B). Because Vero cells are the only cell line used for vaccine production and TMEM236 has broad anti-viral activities (FIG. 13), next tested was the ability to generate a TMEM236 knockout cell line that increases vaccine output. By using the same sgRNA described above, single clonal TMEM236 knockout was generated in Vero cells (FIG. 15). Cells were seeded in T25 flasks and then infected with RV vaccine strains Rotarix or RotaTeq. Knocking out TMEM236 increased the overall virus output by 2 to 10-fold (FIG. 6C, FIG. 6D). Also examined was the propagation of one other vaccine strain in this specific cell line. rVSV-ZEBOV is an Ebola virus vaccine that is constructed on a vesicular stomatitis virus backbone and was recently approved by the FDA. The infectious titer of rVSV-ZEBOV was increased by 4-fold in TMEM236 knockout Vero cells (FIG. 6E). In TMEM236 knockout cells complemented with a V5-tagged TMEM236 (FIG. 16), RotaTeq titer was restored to that in WT cells (FIG. 6F). Taken together, these results indicate that the TMEM236 knockout Vero cell line is a promising cell line for increased vaccine production.

### Discussion

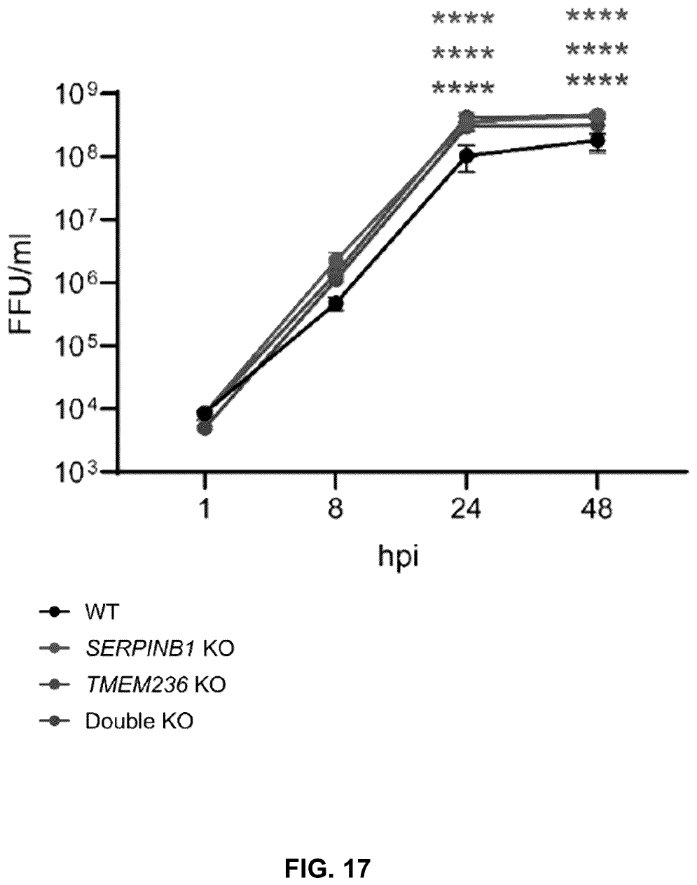

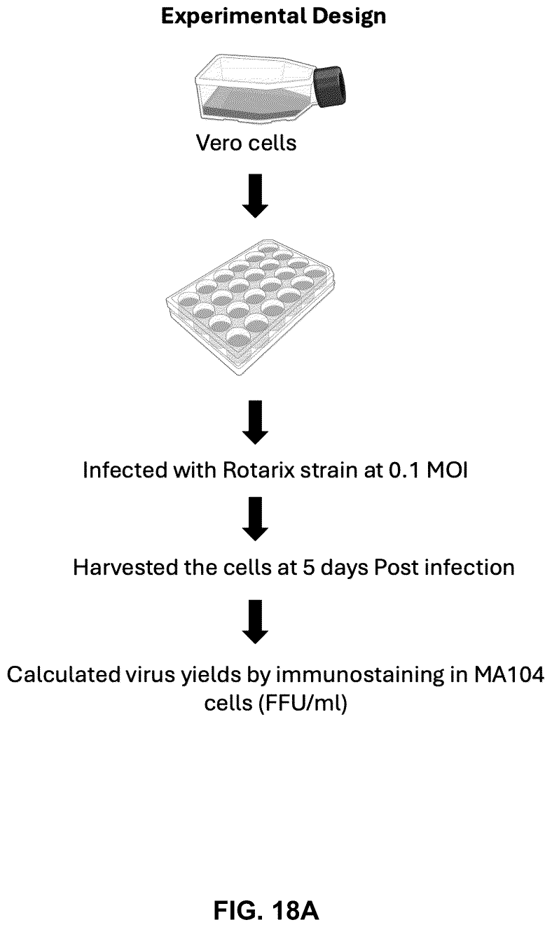

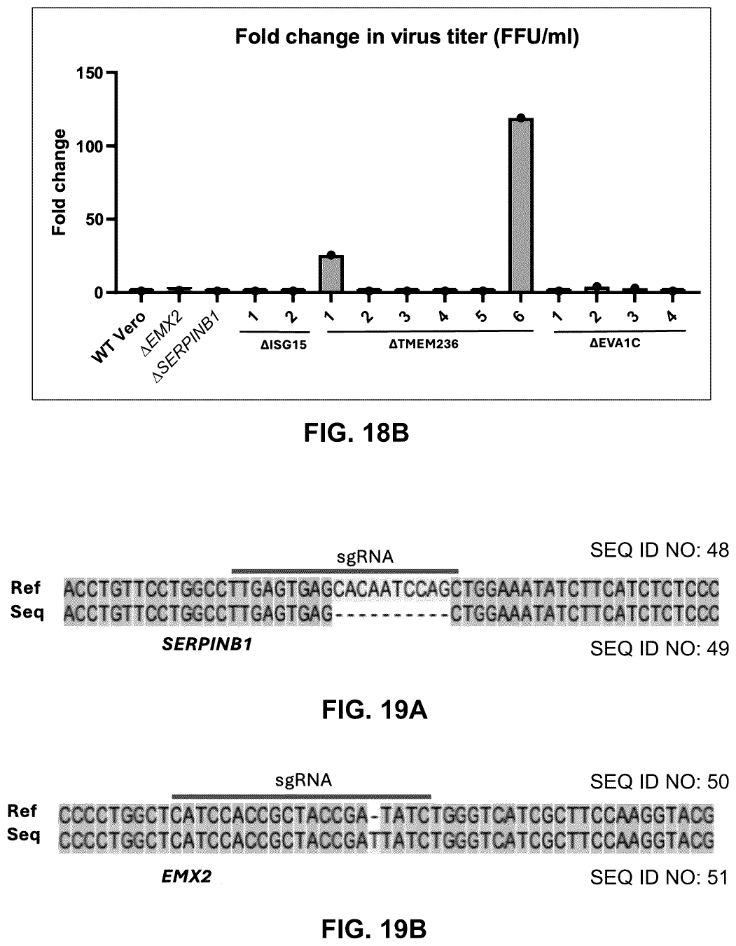

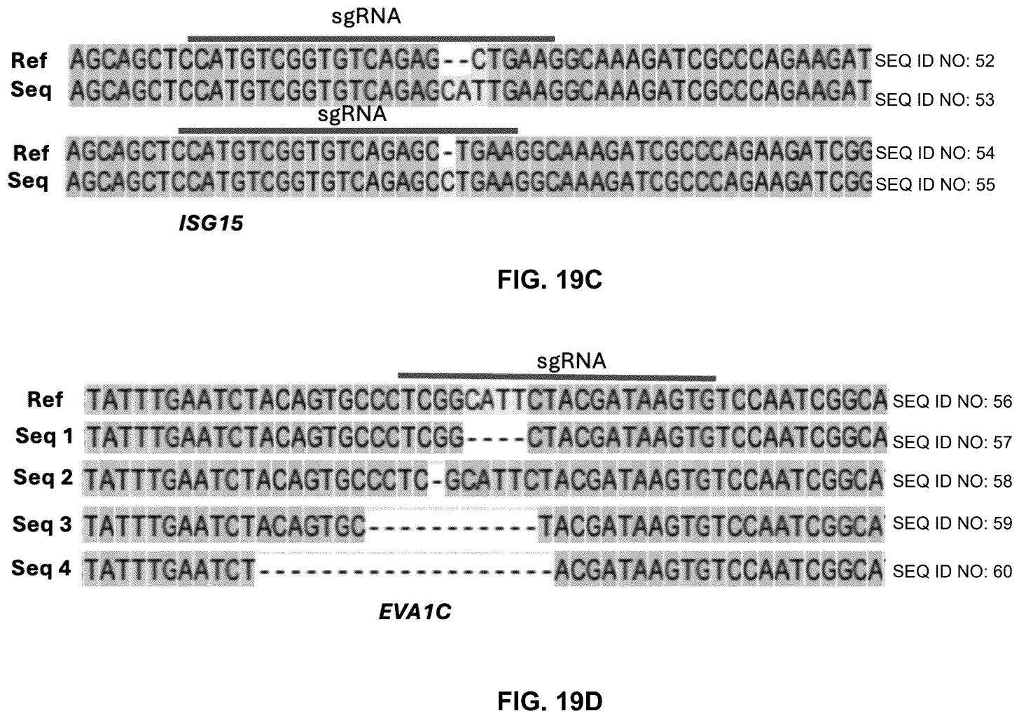

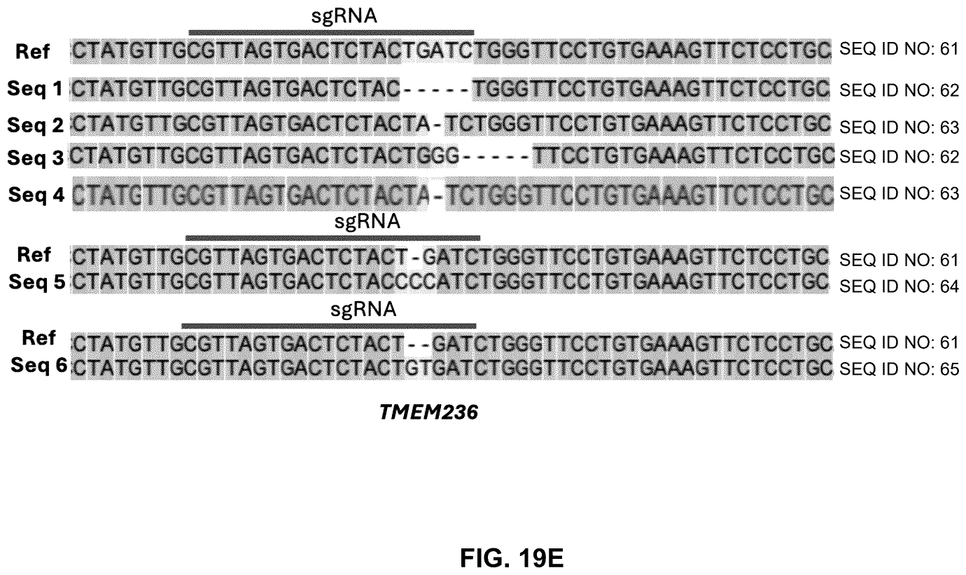

Genome-wide CRISPR/Cas9 gene disruption screens are powerful tools to biological discovery. In this study, FACS-based genome-wide loss-of-function CRISPR/Cas9 screens were performed for a single RV replication cycle, with SERPINB1 and TMEM236 representing the strongest anti-viral genes that inhibited RV replication (FIG. 3A, FIG. 3B). According to information on Human Protein Atlas, SERPINB1 and TMEM236 localize to the cell membrane, which suggests that they may inhibit virus entry. SERPINB1 also localizes to the cytoplasm and lysosome and may block late-stage viral replication. TMEM236 is localized not only to the cell membrane but also to the cytoplasm (FIG. 11). SERPINB1 and TMEM236 double knockouts were generated in MA104 cells, but the virus titer was not altered compared to single gene knockout cells (FIG. 17), suggesting that SERPINB1 and TMEM236 possibly use the same pathway to inhibit RV infection. According to gnomAD, there are at least two pre-mature stop codon mutations and several missense/inframe indels associated with the human TMEM236 gene. The biology of TMEM236 was examined by performing rescue assays and using truncation or point mutants of TMEM236. TMEM236 is enriched in the small intestinal epithelium (FIG. 8B), which contains enterocytes that RV predominantly targets. Therefore, TMEM236 may play a potentially important anti-viral role in vivo. Future generation of Tmem236 knockouts and/or intestinal epithelial cell-conditional knockout mice may prove helpful to evaluate this further.

There are several other interesting leads emerging from this study that also warrant further investigation. Including the able to rescue a murine-like NLuc reporter virus in combination with NSP6 gene deletion with our optimized reverse genetics system. Data from our group and others show that NSP6 deletion has no effect on virus replication in vitro, but it seems to play a role in late-stage viral pathogenesis in vivo (FIG. 5E, FIG. 5F, FIG. 5G, FIG. 5H). The mechanism by which NSP6 deletion limits pathogenesis and potential interaction with the host adaptive immune system will require further study. One previous study shows that SERPINB1 inhibits interferon production and promotes Senecavirus replication. Another study shows that SerpinB1 mitigates inflammation and restricts pro-inflammatory cytokine production during influenza infection in vivo. It was found that opposing results with RRV, an interferon insensitive strain, and rVSV-SARS-CoV-2 (FIG. 3C, FIG. 3D, FIG. 3E, FIG. 13), but further delineating the mechanisms underlying enhanced replication of these strains in the absence of SERPINB1 will benefit from further exploration.

Overall, these newly identified anti-viral factors facilitated the development of a third-generation optimized rotavirus reverse genetics system and revealed gene-edited Vero cells as a rotavirus vaccine substrate for improving vaccine production.

### MATERIALS AND METHODS

### Cell Culture and Viruses

MA104 cells (ATCC CRL-2378) were cultured in Medium 199 (M199, Sigma-Aldrich) supplemented with 10% heat-inactivated fetal bovine serum (FBS), 100 I.U. penicillin/ml, 100 μg/ml streptomycin, and 0.292 mg/ml L-glutamine. BHK-T7 cell line was kindly provided by Dr. Ursula Buchholz (Laboratory of Infectious Diseases, NIAID, NIH, USA) and cultured in completed DMEM supplemented with 0.2 μg/ml of G-418 (Promega) every other passage. MA104 N*V cells were cultured in complete M199 in the presence of 3 μg/ml of puromycin and 3 μg/ml of blasticidin (InvivoGen, San Diego, CA). Vero cells were cultured in DMEM supplemented with 10% FBS.

The recombinant RV strains used in this study include rRRV-GFP, RRV, Rotarix, RotaTeq, UK, WI61, rD6/2-2 g, rD6/2-2g-NSP6-Del-NLuc, and rD6/2-2g-NLuc and were propagated in MA104 cells. Prior to infection, all RV inoculates were activated with 5 μg/ml of trypsin (Gibco Life Technologies, Carlsbad, CA) for 30 min at 37° C. rVSV-SARS-CoV-2 and rVSV-ZEBOV virus were propagated in Vero cells.

### Live/Dead Based Genome-Wide CRISPR-Cas9 Screen

The C. sabaeus genome-wide pooled CRISPR library was used to generate heterogeneous MA104 knockout cell population. A total of 1.6×10⁸ mutagenized cells (0.8×10⁸ cells for both mock and infections) were infected with RRV at a multiplicity of infection (MOI) of 1 for 48 hours. Genomic DNA was harvested from the live cells and sgRNAs were amplified for sequencing on Illumina NextSeq platform. The RIGER algorithm was used for data analysis, taking into account multiple different sgRNAs per gene, number of sequencing reads per gene, and the enrichment of sgRNAs compared to the uninfected pooled library. The core of RIGER analysis was based on gene set enrichment analysis (GSEA) which utilized weighted Kolmogorov-Smirnov (KS) statistics to test whether a predefined set of genes skewed to the top or bottom of the whole gene list.

### FACS-Based Genome-Wide CRISPR-Cas9 Screen

The C. sabaeus genome-wide pooled CRISPR library was used to generate heterogeneous MA104 knockout cell population. A total of 1.6×10⁸ mutagenized cells (0.8×10⁸ cells for both mock and infections) were infected with rRRV-GFP virus at infection (MOI=5) in serum-free medium for 8 h. The 0.1% of GFP positive and GFP negative cells were sorted with flow cytometry. Genomic DNA was harvested from the sorted cells and sgRNAs were amplified for sequencing on Illumina NextSeq platform. The RIGER algorithm was used for data analysis, taking into account multiple different sgRNAs per gene, number of sequencing reads per gene, and the enrichment of sgRNAs compared to the uninfected pooled library.

Protein function analysis was performed using the online Panther Classification System in a three-step manner: 1. uploading IDs of the genes. 2. Choosing Homo sapiens. 3. Selecting Functional classification viewed in graphic charts (Pie). After submission, ontology was selected as Protein Class.

### CRISPR-Cas9 Knockout Cells

Single clonal knockout MA104 and Vero cells were obtained using the PX458 vector that expresses Cas9 and sgRNAs against SERPINB1 (GGCATGCTGAAAATCCACAC; SEQ ID NO: 1) and TMEM236 (CGCAACATAGGCAATAGCAC; SEQ ID NO: 2) (Table 1). GFP positive single cells were sorted at 48 h post-transfection using BD Aria II into 96-well plates and screened for knockout based on Sanger sequencing. Pooled knockout MA104 cells were obtained by lentiviral transduction with the lenti-CRISPR_v2, psPAX2, and pMD2.G vector that expresses Cas9 and sgRNA for a minimum of 14 days under puromycin selection. SERPINB1 gene was amplified with primers, forward primer, ACCACTACTGGAATGCACAGTT (SEQ ID NO: 3); and reverse primer, CAACGTGCAGTGCAGAGTGC (SEQ ID NO: 4). TMEM236 gene was amplified with primers, forward primer, CGAGGACAAGGAACTTGATCCCAG (SEQ ID NO: 5); and reverse primer, CCTGCCTCAGGCTCCCAAAGTGC (SEQ ID NO: 6). Then the DNA was purified with kit and sent for Sanger sequencing.

### Carboxy Fluorescein Succinimidyl Ester (CFSE) Cell Proliferation Assay

MA104 (WT and knockout) cells were stained following a protocol of CFSE Cell Division Tracker Kit (BioLegend, 423801). The cells proliferated and were harvested at 0-96 h post labeling and analyzed by flow cytometry. The intensity of the FITC signal was shown in histogram and normalized to mode.

### Flow Cytometry Analyses

MA104-Cas9 cells were infected with MOI of 5 rRRV-GFP virus for 8 h or MA104 pooled knockouts were infected with MOI of 1 rRRV-GFP for 8 h. Then cells were digested with trypsin and harvested for flow analysis which was described previously. FlowJo v10.10.

### Plaque Assay

Activated virus samples were serially diluted 10-fold and added to monolayers of MA104 cells for 1 h at 37° C. Supernatant was removed and replaced with 0.1% (w/v) agarose (SeaKem® ME Agarose. Lonza) in FBS-free M199 supplement with 0.5 μg/ml of trypsin. Cultures were incubated for 3-4 days at 37° C. in a 5% CO2 incubator. Random plaques were picked by pushing the 200 μL tip through the overlay agarose, and then were propagated in MA104 cells as described above. To quantify the plaque diameter, cultures at 3˜5 dpi were fixed with 10% formaldehyde and stained with neutral red. The diameter of at least 25 randomly selected plaques from 2 independent plaque assays was recorded using an ECHO microscope and then diameters were measured with the annotation tool of the microscope. The images of rVSV-SARS-CoV-2-infected Vero cells were captured by a Typhoon biomolecular imager.

### Focus-Forming Assay

Activated virus samples from cell culture were serially diluted 10-fold and added to confluent monolayers of MA104 cells seeded in 96-well plates for 1 h at 37° C. Inoculates were removed and replaced with M199 serum-free and then incubated for 16-18 h at 37° C. Cells were then fixed with 10% paraformaldehyde and permeabilized with 1% Triton. Cells were incubated with rabbit hyperimmune serum to RRV strain produced in our laboratory and previously described and anti-rabbit HRP-linked secondary antibody. Viral foci were stained with 3-amino-9-ethylcarbazole (AEC substrate kit. Vector Laboratories) per manufacturer's instructions and enumerated visually. Vero cells were infected with rVSV-ZEBOV and GFP positive cells were observed using an ECHO microscope.

### Plasmid Construction

The murine RV rescue plasmids: pT7-D6/2-VP2, pT7-D6/2-VP3, pT7-D6/2-VP4, pT7-D6/2-VP6, pT7-D6/2-VP7, pT7-D6/2-NSP1, pT7-D6/2-NSP2, pT7-D6/2-NSP3, pT7-D6/2-NSP5, pT7-RotaTeq-VP4 (the human RV VP4) and pT7-D6/2-NSP5-NSP6-Del were prepared while pT7-SA11-VP1 and pT7-SA11-NSP4 were originally made by Dr. Takeshi Kobayashi (Research Institute for Microbial Diseases, Osaka University, Japan) and obtained from Addgene. The C3P3-G1 and C3P3-G3 plasmids were kindly provided by Dr. Jais (Eukarys). To generate pT7-D6/2-NSP3-NLuc (accession number: ON738554), which encodes a full-length NLuc gene (GenBank: KM359774.1) and the self-cleaving P2A peptide gene of porcine teschovirus-1, the P2A-NLuc gene cassette was amplified by PCR and inserted between nucleotides in the NSP3 gene via Gibson assembly (NEBuilder HiFi DNA Assembly kit). To generate pT7-D6/2-NSP5-NSP6-Del plasmid, four start codons (sites 1, 25, 60, 65 ATG to ACG) of NSP6 were mutated to other amino acids. Lenti-V5-TMEM236 plasmid was obtained from DNASU (HsCD00936994). Purification of all the plasmids was performed using QIAGEN Plasmid Maxiprep kit per the manufacturer's instructions.

### Generation of Recombinant RVs

rD6/2-2 g was generated using the following pT7 plasmids: pT7-SA11-VP1 and -NSP4, pT7-D6/2-VP2, -VP3, -VP4, -VP6, -VP7, -NSP1, -NSP2, -NSP3 and -NSP5 according to the optimized entirely plasmid-based RG system. The pT7-D6/2-NSP3 plasmid was replaced by the pT7-D6/2-NSP3-NLuc to generate rD6/2-2g-NLuc. The pT7-D6/2-NSP3 plasmid was replaced by the pT7-D6/2-NSP3-NLuc and pT7-D6/2-NSP5 plasmid was replaced by the pT7-D6/2-NSP5-NSP6-Del combined with C3P3-G3 plasmid and overlayed with SERPINB1 knockout MA104 cells to generate rD6/2-2g-NSP6-Del-NLuc virus. rSA11-RotaTeq-VP4 was generated using the following pT7 plasmids: pT7-SA11-VP1, VP2, -VP3, -VP6, -VP7, -NSP1, -NSP2, -NSP3, -NSP4 and -NSP5. The pT7-SA11-VP4 plasmid was replaced by the pT7-RotaTeq-VP4 plasmid to generate rSA11-RotaTeq-VP4 virus. The rescued recombinant RVs were propagated for two passages in MA104 cells in a 6-well plate, and then were plaque purified twice in MA104 cells.

The optimized protocol is described as follows: 2×10⁵ BHK-T7 cells were seeded into 1 well of 12-well plate with 1 ml of complete DMEM (10% heat-inactivated FBS, 100 IU/ml penicillin, 100 μg/ml streptomycin, 0.292 mg/ml) G418-free medium. Twenty four hours later, the medium was replaced by 800 μL of fresh complete DMEM medium, and then the subconfluent BHK-T7 monolayer was transfected with the corresponding transfection mix, which contained 125 μL of prewarmed Opti-MEM, 400 ng each of the 8 RV pT7 plasmid, pT7-VP1-7, pT7-NSP1,3,4 and 1200 ng pT7-NSP2,5, and 800 ng of the plasmid C3P3-G3. Then added 14 μL of TransIT-LTI (Mirus Bio LLC). All the plasmids and transfection reagents were mixed in a pipet by gently moving them up and down and then incubated at room temperature for 15 min. Transfection mixture was added drop by drop to the medium of BHK-T7 monolayers, and then the cells were returned to 37° C. 18 h later, two washes with FBS-free medium, after that 800 μL of serum-free DMEM was added to the transfected-BHK-T7 cells. Twenty-four hours later, 5×10⁴ SERPINB1 knockout MA104 in 200 μL of serum-free DMEM was added to the well, along with 0.5 μL/mL of porcine pancreatic type IX-S trypsin (Sigma-Aldrich). SERPINB1 knockout MA104 and BHK-T7 cells were cocultured for 72 h, after which they were frozen and thawed three times. To remove cell debris, the lysate was centrifuged at 350×g for 10 min at 4° C. and then activated with 2.5 μg/ml of trypsin to infect a three-day-old monolayer of MA104 cells. After 1 h of adsorption, the inocula were removed, and 1 ml of serum-free 199 medium supplemented with 0.5 μg/ml of trypsin was placed on the cells. MA104 cells were incubated at 37° C. for 5 days or until cytopathic effects were observed (passage 1). Successfully rescued cells were defined as when MA104 cells infected with the corresponding RV rescued passage 1 were positive by immunostaining using an anti-double-layered particle antibody.

### RT-qPCR

The total RNA of the MA104 cells infected with recombinant RRV, rD6/2-2 g, rD6/2-2g-NLuc, and rD6/2-2g-NSP6-Del-NLuc virus were extracted by TRIzol. Total RNA was reverse transcribed to cDNA using a high-capacity cDNA reverse transcription kit with RNase inhibitor (Applied Biosystems) according to the user guide. Briefly, 0.8 μg of RNA, 2 μL of 10× reverse transcription (RT) buffer, 0.8 μL of 100 mM deoxynucleoside triphosphate (dNTP) mix, 2 μL of RT random primers, 0.1 μL of RNase inhibitor, 0.1 μL of MultiScribe reverse transcriptase, and a flexible amount of nuclease-free H₂O were added to the 20 μL reaction mixture. The reverse transcription thermocycling program was set at 25° C. for 10 min, 37° C. for 2 h, and 85° C. for 5 min. The expression level of housekeeping gene GAPDH was quantitated by 2×SYBR green master mix (Applied Biosystems), and NSP5 was quantitated by 2×TaqMan Fast Advanced master mix (Applied Biosystems). The primers used in this study were as follows: human GAPDH forward primer, 5′-GGAGCGAGATCCCTCCAAAAT-3′ (SEQ ID NO: 7), and reverse primer, 5′-GGCTGTTGTCATACTTCTCATGG-3′ (SEQ ID NO: 8); SERPINB1 forward primer, 5′-AAGGAGCTCAGCATGGTCA-3′ (SEQ ID NO: 9); and reverse primer, 5′-GGGCGAGGTCTGAGTTGAGG-3′ (SEQ ID NO: 10); TMEM236 forward primer, 5′-CCTGACCTACCCGTGTCTCTGG-3′ (SEQ ID NO: 11); and reverse primer, 5′-ACCAGCCACACGCACCATTT-3′ (SEQ ID NO: 12); and NSP5 forward primer, 5′-CTGCTTCAAACGATCCACTCAC-3′ (SEQ ID NO: 13), reverse primer, 5′-TGAATCCATAGACACGCC-3′ (SEQ ID NO: 14), and probe, 5′-CY5/TCAAATGCAGTTAAGACAAATGCAGACGCT/IABRQSP-3′ (SEQ ID NO: 15). The y axis stands for the percentage of NSP5 mRNA levels relative to GAPDH levels.

### Luciferase Assay

MA104 cells seeded in 96-well plates were infected with 50 μL of 10-fold serial dilution of recombinant RVs at 37° C. for 48 h and freeze-thawed 2 times before 50 μL/well of Nano-Glo Luciferase Assay Reagent (Promega) was added per manufacturer's instructions. After 5 minutes incubation at room temperature, relative luminosity units were measured (p/sec/cm2/sr) using a 20/20n Luminometer (Turner Biosystems).

### Purification of RV Particles by Sucrose Gradient Centrifugation

RVs were concentrated by pelleting through a sucrose cushion. Briefly, MA104 grown in 12-well plate were infected at an MOI of 0.01 and harvested at 72 h post infection (hpi), the viral lysates were freeze-thawed three times, and viral particles concentrated by ultracentrifugation for 1 h at 30,000 g at 4° C. Viral pellets were resuspended in TNC buffer (10 mM Tris/HCl [pH 7.5], 140 mM NaCl, 10 mM CaCl2), extracted with genetron and the aqueous phase pelleted through a 40% sucrose cushion by centrifugation for 1 h at 30,000g at 4° C. The pelleted RV was resuspended with 1 mL of PBS with 100 mg/L of Ca²⁺ and Mg²⁺ and this suspension was used to perform MA104-Cas9 cells infection or to obtain genomic dsRNA profiles.

Electrophoresis of Viral dsRNA Genomes

Viral dsRNAs were extracted from sucrose cushion-concentrated RVs with TRIzol (Invitrogen) according to the manufacturer's protocol and then mixed with Gel Loading Dye, Purple (6×), no SDS (NEB). Samples were subjected to PAGE (10%) for 2 h 30 min at 180 V and then stained with ethidium bromide (0.1 μg/mL) for 10 minutes and visualized by the gel documentation system (Axygen).

### Immunofluorescence Analysis

MA104 cells were transfected with indicated plasmids for 48 hours with Lipofectamine 3000 (Invitrogen) according to the manufacturer's protocol. Cells were washed three times with phosphate-buffered saline (PBS) and then fixed with 4% (wt/vol) paraformaldehyde for 15 min at room temperature. Cells were then washed three times with PBS and incubated with 0.1% Triton X-100 for 10 min. Next, 5% bovine serum albumin was used to block for 2 h. Cells were then incubated in Alexa Fluor FITC-conjugated goat anti-rabbit (V5) secondary antibodies for 2 h. Nuclei were stained with DAPI (00-4959-52, Invitrogen) for 10 min. All cells were washed with PBS 5 times after each step and were imaged by microscopy.

### Western Blot

Vero cells expressing V5-TMEM236 were washed with cold phosphate-buffered saline (PBS) and lysed in cell lysis buffer for western blotting containing protease inhibitor cocktail (04693132001, Roche, Basel, Switzerland) by using a rabbit anti-V5 antibody (Cell Signaling Technology).

### In Vivo Imaging System (IVIS)

C57BL/6 mice were purchased from the Jackson Laboratory and bred locally at the Washington University in St. Louis (WUSTL) CSRB vivarium. Five-day-old suckling pups were orally infected with rD6/2-2g-NLuc and rD6/2-2g-NSP6-Del-NLuc viruses (3.5×10³ FFU). Diarrhea was evaluated from day 1 to day 8 post infection. To perform IVIS, first the mice were weighed, and oral gavage Nano-Glo™ substrate (1/20 dilution in PBS; to make sure 50 μL per mouse, 1/25-1/57 dilution in PBS) for 3.5 hours and then performed IVIS (exposure time: 1 second) by using the IVIS Spectrum BL.

### Statistical Analysis

All statistical tests were performed as described in the indicated figure legends using GraphPad Prism. Statistical significance was determined using a one-way or two-way ANOVA Tukey's post hoc test when comparing three or more groups. Attest was performed to compare with two groups. The number of 5 independent experiments performed is indicated in the relevant figure legends.

TABLE 1
sgRNA gene sequences used in the generation of knockout cells.

| SEQ ID NO | Name | Sequence |
|---|---|---|
| SEQ ID NO: 16 | SERPINB1sgRNAF | CACCGGGCATGCTGAAAATCCACAC |
| SEQ ID NO: 17 | SERPINB1sgRNAR | AAACGTGTGGATTTTCAGCATGCCC |
| SEQ ID NO: 18 | RHOBsgRNAF | CACCGCAGTAAGGACGAGTTCCCCG |
| SEQ ID NO: 19 | RHOBsgRNAR | AAACCGGGGAACTCGTCCTTACTGC |
| SEQ ID NO: 20 | PDE4CsgRNAF | CACCGGGGACTTGACGTGTTCAAGG |
| SEQ ID NO: 21 | PDE4CsgRNAR | AAACCCTTGAACACGTCAAGTCCCC |
| SEQ ID NO: 22 | GPR22sgRNAF | CACCGTGATAGTGGTTGGTACATGT |
| SEQ ID NO: 23 | GPR22sgRNAR | AAACACATGTACCAACCACTATCAC |
| SEQ ID NO: 24 | ADRB1sgRNAF | CACCGCACGCACAGCACGTCCACCG |
| SEQ ID NO: 25 | ADRB1sgRNAR | AAACCGGTGGACGTGCTGTGCGTGC |
| SEQ ID NO: 26 | FAM8A1sgRNAF | CACCGGAACGGGAGCGCCGCCCGAA |
| SEQ ID NO: 27 | FAM8A1sgRNAR | AAACTTCGGGCGGCGCTCCCGTTCC |
| SEQ ID NO: 28 | KBTBD4sgRNAF | CACCGTCGGTCACACTCAGGCCGTG |
| SEQ ID NO: 29 | KBTBD4sgRNAR | AAACCACGGCCTGAGTGTGACCGAC |
| SEQ ID NO: 30 | TMEM236sgRNAF | CACCGCGCAACATAGGCAATAGCAC |
| SEQ ID NO: 31 | TMEM236sgRNAR | AAACGTGCTATTGCCTATGTTGCGC |
| SEQ ID NO: 32 | CD28sgRNAF | CACCGCATGTGCCTTAAGGGGATGG |
| SEQ ID NO: 33 | CD28sgRNAR | AAACCCATCCCCTTAAGGCACATGC |
| SEQ ID NO: 34 | SLC7A6sgRNAF | CACCGGGACACGTTCACTTACGCCA |
| SEQ ID NO: 35 | SLC7A6sgRNAR | AAACTGGCGTAAGTGAACGTGTCCC |
| SEQ ID NO: 36 | ALAS1sgRNAF | CACCGTGGATGAGGTCCATGCAGTG |
| SEQ ID NO: 37 | ALAS1sgRNAR | AAACCACTGCATGGACCTCATCCAC |
| SEQ ID NO: 38 | SPATA4sgRNAF | CACCGTCACGGACTATAGTTACCAG |
| SEQ ID NO: 39 | SPATA4sgRNAR | AAACCTGGTAACTATAGTCCGTGAC |

What is claimed is:

1. A rotavirus reverse genetics system, the system comprising a SERPINB1 knockout cell or a TMEM236 knockout cell.

2. The system of claim 1, wherein the system further comprises:
a. a helper plasmid comprising a C3P3-G3 plasmid;
b. a layer of BHK-T7 cells; and
c. any combination thereof.

3. The system of claim 1, wherein the SERPINB1 knockout cell or the TMEM236 knockout cell comprises a Vero cell.

4. The system of claim 1, wherein the SERPINB1 knockout cell or TMEM236 knockout cell are generated using a CRISPR/Cas9 system comprising a single-guide RNA (sgRNA) selected from SEQ ID NO: 1 and SEQ ID NO: 2.

5. The system of claim 1, wherein the SERPINB1 knockout cell or the TMEM236 knockout cell are infected with a live-attenuated vaccine.

6. The system of claim 5, wherein the live-attenuated vaccine comprises a Rotavirus vaccine or an Ebola vaccine.

7. A vaccine-producing cell substrate, the substrate comprising a Vero cell with disrupted SERPINB1 or TMEM236.

8. The substrate of claim 7, wherein the Vero cells are transfected with a CRISPR/Cas9 plasmid comprising a sgRNA selected from SEQ ID NO: 1 or SEQ ID NO: 2, to disrupt SERPINB1 or TMEM236.

9. The substrate of claim 7, wherein the vaccine-producing cell substrate increases production of a live-attenuated vaccine.

10. The substrate of claim 9, wherein the live-attenuated vaccine comprises a rotavirus (RV) vaccine or an Ebola vaccine.

11. A method of increasing production of a vaccine in a cell, the method comprising:
a. disrupting an anti-viral protein in the vaccine producing cell;
b. introducing a helper component to the vaccine producing cell;
c. infecting the vaccine producing cells with a live-attenuated vaccine; and
d. collecting the vaccine from the vaccine producing cell.

12. The method of claim 11, wherein the vaccine producing cell is a Vero cell.

13. The method of claim 11, wherein the anti-viral protein disrupted in the vaccine producing cell is TMEM236 or SERPINB1.

14. The method of claim 13, wherein TMEM236 or SERPINB1 is knocked out of the vaccine producing cell using a CRISPR/Cas9 system comprising a sgRNA selected from SEQ ID NO: 1 and SEQ ID NO: 2.

15. The method of claim 11, wherein the helper component comprises a C3P3-G3 plasmid, a BHK-T7 cell, or any combination thereof.

16. The method of claim 11, wherein the live-attenuated vaccine comprises a Rotavirus vaccine or an Ebola vaccine.

17. The method of claim 11, wherein vaccine production is increased by 2-fold to 10-fold.
